(19) United States
(12) Patent Application Publication
(10) Pub. No.: US 2003/0087313 A1
(43) Pub. Date: May 8, 2003

(54) Dose response-based methods for identifying receptors having alterations in signaling

(76) Inventors: Alan S. Kopin, Wellesley, MA (US); Martin Beinborn, Boston, MA (US)

Correspondence Address: CLARK & ELBING LLP, 101 FEDERAL STREET, BOSTON, MA, 02110, US

(21) Appl. No.: 10/139,084

(22) Filed: May 3, 2002

Related U.S. Application Data
(60) Provisional application No. 60/288,647, filed on May 3, 2001.

(51) Int. Cl. G01N 33/53; G01N 33/567; C12N 5/06; C07K 14/705; C12N 15/85

(52) U.S. Cl. 435/7.2; 435/455; 435/320.1; 435/325; 530/350

(57) ABSTRACT

The invention provides methods of identifying receptors having altered signaling. In particular, the invention provides a sensitive dose response assay for the identification of receptors having alterations in ligand dependent or ligand independent signaling.

### CROSS REFERENCE TO RELATED APPLICATIONS

This application claims the benefit of the filing date of U.S. provisional application, U.S. S. No. 60/288,647, filed May 3, 2001.

### STATEMENT AS TO FEDERALLY SPONSORED RESEARCH

This application was supported in part by NIH grant DK46767. The government may have certain rights to this invention.

### FIELD OF THE INVENTION

In general, the invention provides methods for the identification of receptors having altered signaling.

### BACKGROUND OF THE INVENTION

Receptors having altered signaling, for example, constitutively active, hypersensitive, hyposensitive, silenced, or non-functional receptors, can be important tools for drug discovery given their role in the etiology of diseases or pathological conditions in humans and animals. The identification of receptors having altered signaling is also valuable in the identification of polymorphic receptors where the altered signaling contributes to health or disease. Similarly, it is important to identify mutant or polymorphic receptors where the mutation or polymorphism alters the response of the receptor to a particular ligand, for example, a drug or peptide hormone.

Receptor activity has been typically measured by assaying induction of intracellular second messenger signals, or by employing standard transcriptional reporter assays. Sensitive methods of identifying receptors having mutation or polymorphism-induced alterations in signaling have however been lacking. For example, the identification of receptors having alterations in basal signaling, such as constitutively active receptors, has posed particular challenges. It would be useful to have sensitive assays for the identification of receptors having altered signaling.

### SUMMARY OF THE INVENTION

The invention generally provides methods of identifying receptors having altered signaling. In particular, the invention provides a sensitive dose response assay for the identification of receptors having alterations in ligand dependent or ligand independent signaling.

In one aspect, the invention provides a method of identifying a receptor with altered signaling, by co-transfecting a first host cell with an expression vector, where the expression vector includes a promoter operably linked to a candidate receptor, and a reporter construct, where the reporter construct includes a response element and a promoter operably linked to a reporter gene, the response element being sensitive to a signal induced by the receptor; co-transfecting a second host cell with the reporter construct and a negative control vector; measuring the level of expression of the reporter construct in the first host cell and in the second host cell at varying concentrations of the reporter construct or at varying concentrations of the expression vector or the negative control vector, such that dose-response curves are generated for the expression of the reporter construct in the first and the second host cells; and identifying the candidate receptor as a receptor with altered signaling by its ability to increase or decrease the level of expression in the first host cell compared to the level of expression in the second host cell over a range of at least two different concentrations of the reporter construct, the negative control vector, or the expression vector.

In an embodiment of the this aspect, the reporter construct may include a luciferase construct, a beta-galactosidase construct, or a chloramphenicol acetyl transferase construct. In another embodiment of this aspect, the response element may include the somatostatin promoter, the serum response element, or the cAMP response element. In yet another embodiment of this aspect, the receptor with altered signaling can be a constitutively active receptor, a hypersensitive receptor, a hyposensitive receptor, a non-functional receptor, a silent receptor, a partially silent receptor, a transmembrane receptor, a nuclear receptor, a steroid hormone receptor, a mutant receptor, a polymorphic receptor, or a G protein coupled receptor. The G protein-coupled receptor can be coupled to a G protein, for example, Gαq, Gαs, Gαi, and Go.

In another embodiment of this aspect, the method can further include co-transfecting the first host cell with a second expression vector, the second expression vector comprising a promoter operably linked to a chimeric G protein, wherein the chimeric G protein is capable of receiving a signal from the G protein-coupled receptor and increasing the expression of the reporter construct; and co-transfecting the second host cell with the second expression vector. The chimeric G protein can be Gq5i, Gq5o, Gq5z, Gq5s, Gs5q, or G13Z.

In other embodiments of this aspect, the range is over at least three different concentrations of the reporter construct or the expression vector, or over at least five different concentrations of the reporter construct or the expression vector.

In other embodiments of this aspect, the signaling can be ligand dependent signaling or ligand independent signaling. In another embodiment of this aspect, the receptor with altered signaling can be further screened for an alteration in a response induced by a ligand. The ligand can be a drug, an agonist, an antagonist, or an inverse agonist.

In another aspect, the invention provides a method of identifying a G protein-coupled receptor with altered signaling, by co-transfecting a first host cell with a reporter construct, the reporter construct including a G protein response element and a promoter operably linked to a reporter gene, a first expression vector, the first expression vector including a promoter operably linked to a candidate G protein-coupled receptor, and a second expression vector, the second expression vector including a promoter operably linked to a chimeric G protein, where the chimeric G protein is capable of receiving a signal from the candidate G protein-coupled receptor and increasing the expression of the reporter construct; co-transfecting a second host cell with the reporter construct, the second expression vector, and a negative control vector; and measuring the level of expression of the reporter construct in the first host cell and the second host cell, where an increased or decreased level of expression in the first host cell compared to the second host cell identifies the candidate receptor as a G protein-coupled receptor with altered signaling.

In an embodiment of this second aspect, the chimeric G protein includes a G protein with the C-terminal 3 amino acids changed to those of another G protein. In another embodiment of this second aspect, the chimeric G protein can be Gq5i, Gq5o, Gq5z, Gq5s, Gs5q, or G13Z. The reporter construct can be a luciferase construct, a beta-galactosidase construct, or a chloramphenicol acetyl transferase construct. The response element can be the somatostatin promoter, the serum response element, or the cAMP response element.

In other embodiments of the invention, the G protein coupled receptor can be a constitutively active receptor, a hypersensitive receptor, a hyposensitive receptor, a non-functional receptor, a silent receptor, or a partially silent receptor. In other embodiments of the invention, the G protein-coupled receptor can be coupled to a G protein, for example, Gαq, Gαs, Gαi, or Go. The signaling can be ligand dependent signaling or ligand independent signaling. In another embodiment of this aspect, the receptor with altered signaling can be further screened for an alteration in a response induced by a ligand. The ligand can be a drug, an agonist, an antagonist, or an inverse agonist.

The methods for detecting receptors with altered signaling, described herein, are applicable in the detection of many kinds of altered signaling. For example, the methods are capable of detecting receptors having an increase or decrease in basal signaling, receptors having an increased or decreased sensitivity to ligand stimulation, receptors having increased or decreased potency, and even receptors that do not transmit a signal. The invention is particularly valuable because it has the ability to rapidly and reproducibly identify mutant and/or polymorphic receptors having such alterations in activity. Such mutant and polymorphic receptors having such alterations include G protein-coupled receptors (for example, G protein-coupled receptors coupled to Gq, Gs, Gi, or Go proteins), transmembrane receptors, and nuclear receptors (for example, steroid hormone receptors). Once identified, such receptors can be further screened for an alteration in a ligand induced response, for example, an altered response to a drug.

The particular response element used in the assay of the invention may be any response element that is sensitive to signaling through a particular receptor. Examples of preferred response elements include a portion of the somatostatin promoter (SMS), which includes a number of different response elements, the serum response element (SRE), and the cAMP response element (CRE), which are sensitive to G protein-coupled receptor signaling. Other response elements include those sensitive to signaling through a single transmembrane receptor or a nuclear receptor. The signaling detected by a particular response element can be any of the types of receptor signaling discussed herein, including increased basal signaling (constitutive signaling), decreased basal signaling (full or partial silencing), and hypersensitive or hyposensitive signaling.

As used herein, by “altered signaling” is meant a change in the ligand dependent or ligand independent signal typically generated by a receptor, as measured by the parameters of efficacy, potency, or basal signaling. The change or alteration may be an increase or decrease in ligand dependent or ligand independent signaling. Examples of alterations in signaling include receptors having an increased sensitivity to ligand, i.e., hypersensitive receptors. This increased sensitivity to ligand may occur in the form of increased potency or increased efficacy in response to agonist stimulation. Other examples of receptors having alterations in signaling include receptors exhibiting a decreased sensitivity to ligand (i.e., hyposensitive or silenced receptors), receptors exhibiting a change in basal activity (e.g., receptors having an increased level of basal signaling, such as constitutively active receptors, or receptors having a decreased level of basal signaling, such as receptors having silencing mutations, i.e., fully silenced or partially silenced receptors). The change or alteration in signaling may also be an absence of signaling, for example, a non-functional receptor that does not bind a ligand, or a receptor that binds a ligand but does not transduce a ligand induced signal. A receptor with altered signaling exhibits at least a 25% increase or decrease in basal activity, or at least a 50% increase or decrease in basal activity, or at least a 75% increase or decrease in basal activity, or more than a 100% increase or decrease in basal activity, compared to an appropriate negative control. Alternatively, or in addition, a receptor with altered basal signaling exhibits at least a 5% increase or decrease, or at least a 10%, 15%, 20%, or 25% increase or decrease, or at least a 50%, 60%, or 75% increase or decrease, or more than a 100% increase or decrease in basal activity when expressed as a percentage of the hormone-induced maximal activity, all compared to an appropriate negative control. At the very least, a receptor with altered signaling exhibits a change in basal or ligand induced signaling or efficacy or potency relative to an appropriate negative control that is considered statistically significant using accepted methods of statistical analysis.

“Basal” activity means the level of activity (e.g., activation of a specific biochemical pathway or second messenger signaling event) of a receptor in the absence of stimulation with a receptor-specific ligand (e.g., a positive agonist). In many cases, the basal activity is less than the level of ligand-stimulated activity of a wild-type receptor. However, in certain cases, a receptor with increased basal activity may display a level of signaling that approximates, is equal to, or exceeds the level of ligand-stimulated activity of the corresponding wild type receptor.

“Expression vectors” contain at least a promoter operably linked to the gene to be expressed. “Promoter” means a minimal sequence sufficient to direct transcription. Also included in the invention are those promoter elements which are sufficient to render promoter-dependent gene expression controllable for cell-type specificity, tissue-specificity, or induction by external signals or agents; such elements may be located in the 5′ or 3′ regions of the native gene. A promoter element may be positioned for expression if it is positioned adjacent to a DNA sequence so it can direct transcription of the sequence. “Operably linked” means that a gene and a regulatory sequence(s) are connected in such a way as to permit gene expression when the appropriate molecules (e.g., transcriptional activator proteins) are bound to the regulatory sequence(s).

A “reporter construct” includes at least a promoter operably linked to a reporter gene. Such reporter genes may be used in any assay for measuring transcription or translation and may be detected directly (e.g., by visual inspection) or indirectly (e.g., by binding of an antibody to the reporter gene product or by reporter product-mediated induction of a second gene product). Examples of standard reporter genes include genes encoding luciferase, green fluorescent protein (GFP), or chloramphenicol acetyl transferase (see, for example, Sambrook, J. et al., Molecular Cloning: a Laboratory Manual, Cold Spring Harbor Press, N.Y., or Ausubel et al., Current Protocols in Molecular Biology, Greene Publishing Associates, New York, N.Y., V 1-3, 2000, incorporated herein by reference). Expression of the reporter gene is detectable by use of an assay that directly or indirectly measures the level or activity of the reporter gene. Preferred reporter constructs also include a response element.

A “response element” is a nucleic acid sequence that is sensitive to a particular signaling pathway, e.g., a second messenger signaling pathway, and assists in driving transcription of the reporter gene. According to the present invention, the response element may refer to a promoter that is activated in response to signaling through a particular receptor. “Second messenger signaling activity” refers to production of an intracellular stimulus (including, but not limited to, cAMP, cGMP, ppGpp, inositol phosphate, or calcium ions) in response to activation of the receptor, or to activation of a protein in response to receptor activation, including but not limited to a kinase, a phosphatase, or to activation or inhibition of a membrane channel.

A “negative control,” as used herein, is any construct that can be used to distinguish alterations in the signaling of a candidate receptor. The appropriate negative control for any given candidate receptor will vary depending on the assay and the type of alteration in signaling. For example, to identify a constitutively active receptor, the appropriate negative controls may be a vector lacking any receptor nucleotide sequences, a vector including non-constitutively active wild type receptor nucleotide sequences, or a vector including silenced receptor nucleotide sequences. Alternatively, to identify a silenced receptor, the appropriate negative controls may a vector including wild type receptor nucleotide sequences, or a vector including constitutively active receptor nucleotide sequences. The appropriate negative control to be used to identify a receptor with altered signaling will be apparent to a person of ordinary skill in the art.

An “agonist,” as used herein, is a chemical substance that interacts with a receptor to initiate a function of the receptor. For example, for peptide hormone receptors, the agonist preferably alters a second messenger signaling activity. A positive agonist is a compound that enhances or increases the activity or second messenger signaling of a receptor. A “full agonist” refers to an agonist capable of activating the receptor to the maximum level of activity, e.g., a level of activity that is substantially equivalent to that level induced by a natural ligand, e.g., an endogenous peptide hormone. A “partial agonist” refers to a positive agonist with reduced intrinsic activity relative to a full agonist. As used herein, a “peptoid” is a peptide-derived partial agonist. An “inverse agonist,” as used herein, has a negative intrinsic activity, and reduces the receptor's signaling activity relative to the signaling activity measured in the absence of the inverse agonist (see also Milligan et al., TIPS, 16:10-13, 1995). By contrast, “antagonist” refers to a chemical substance that inhibits the ability of an agonist to increase or decrease receptor activity. A ‘neutral’ or ‘perfect’ antagonist has no intrinsic activity, and no effect on the receptor's basal activity. Peptide-derived antagonists are, for the purposes herein, not distinguished from non-peptide ligands.

### BRIEF DESCRIPTION OF THE DRAWINGS

FIG. 1 is a dose response curve of wild type and mutant CCK-2 receptor and a negative control co-transfected with 5 ng SRE-Luc reporter construct.

FIGS. 2A-B are two examples, from independent experiments, of dose response curves of wild type and mutant CCK-2 receptor and a negative control co-transfected with 35 ng SRE-Luc reporter construct.

FIG. 3 is a dose response curve of wild type and mutant CCK-2 receptor and a negative control co-transfected with 150 ng SRE-Luc reporter construct.

FIGS. 4A-B are two examples, from independent experiments, of dose response curves of wild type and mutant MC-4 receptor and a negative control co-transfected with 35 ng Sms-Luc reporter construct.

FIG. 5 is a dose response curve of wild type and two mutant PTH receptors and a negative control co-transfected with 35 ng Sms-Luc reporter construct.

FIGS. 6A-B are two examples, from independent experiments, of dose response curves of wild type and mutant mu opioid receptor and a negative control co-transfected with 35 ng SRE-Luc reporter construct and 7 ng Gq5i.

FIG. 7 is a bar graph of a first, constitutively active MC4 receptor co-transfected with Sms-Luc reporter as well as various second receptors or negative controls.

FIG. 8 is a dose response curve of a first, constitutively active MC4 receptor co-transfected with Sms-Luc reporter as well as various second receptors or a negative control.

FIG. 9 is a table of constitutively active Class I G protein-coupled receptors, which have increased basal activity. The amino acids that, when mutated, impart constitutive activity to the receptors are indicated.

### DETAILED DESCRIPTION OF THE INVENTION

Receptors with altered signaling are functionally abnormal receptors, compared to the corresponding wild-type receptor, and can serve as efficient screens for agonist drugs by effectively lowering the threshold for receptor activation. For example, an increase in the basal activity of a receptor (i.e., a constitutively active receptor) allows the detection of agonist activity that would not otherwise be identified using the naturally occurring wild-type receptor. In addition, an inverse agonist can be detected using constitutively active receptors due to drug induced inhibition of the (increased) basal activity which would not be apparent in a non-constitutively active receptor. Similarly, a decrease in the basal activity of a receptor (i.e., a silenced or partially silenced receptor) allows the detection of agonist activity that would otherwise be masked by a high level of basal background activity. For the same reason, silenced or partially silenced receptors also provide better detection of neutral antagonists as defined by inhibition of agonist-induced signaling. Receptors with altered signaling therefore provide a more sensitive screen for drug discovery. The invention provides rapid, sensitive, and reproducible screening assays for the detection of alterations in the signaling activity of a receptor.

The screening assays of the invention can be applied to receptors with known ligands, as well as to receptors for which the ligand is presently unknown (e.g., orphan receptors). Any of the ligands identified using a receptor with altered signaling may, upon further experimentation, prove to be a useful therapeutic agent. Such therapeutic agents may be used to treat or prevent a disease or disorder, or improve the health of an individual.

Receptors with Altered Signaling

Receptors with altered signaling include constitutively active receptors, hypersensitive receptors, hyposensitive receptors, non-functional receptors, and fully or partially silenced receptors. These receptors may be naturally occurring, polymorphic, or mutant.

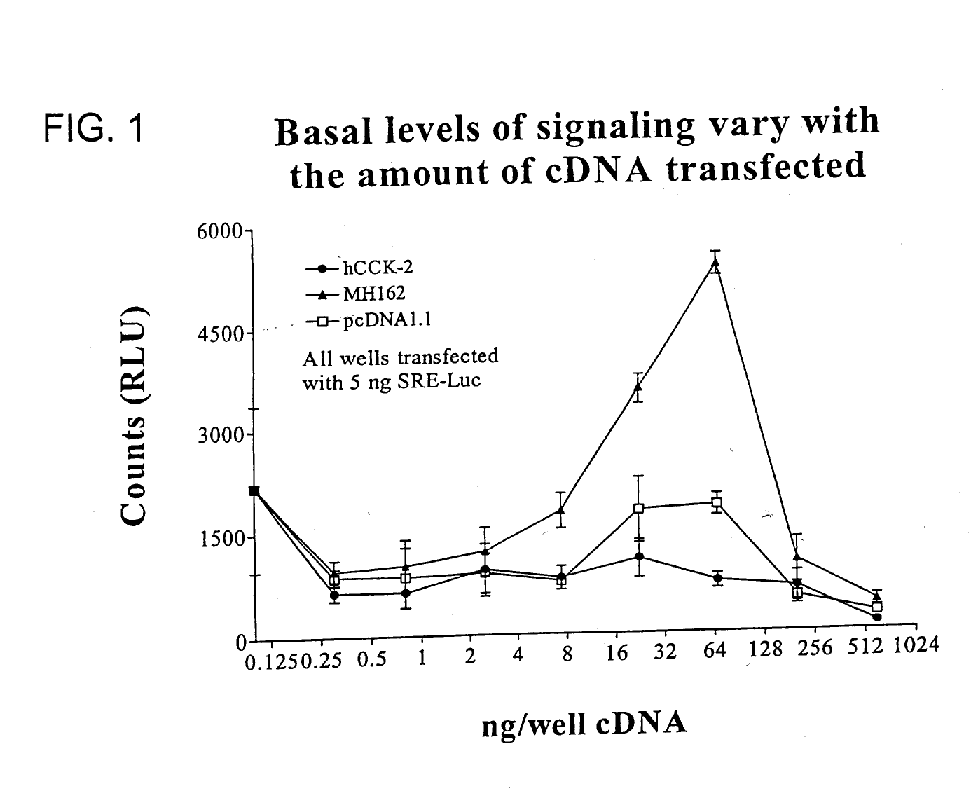

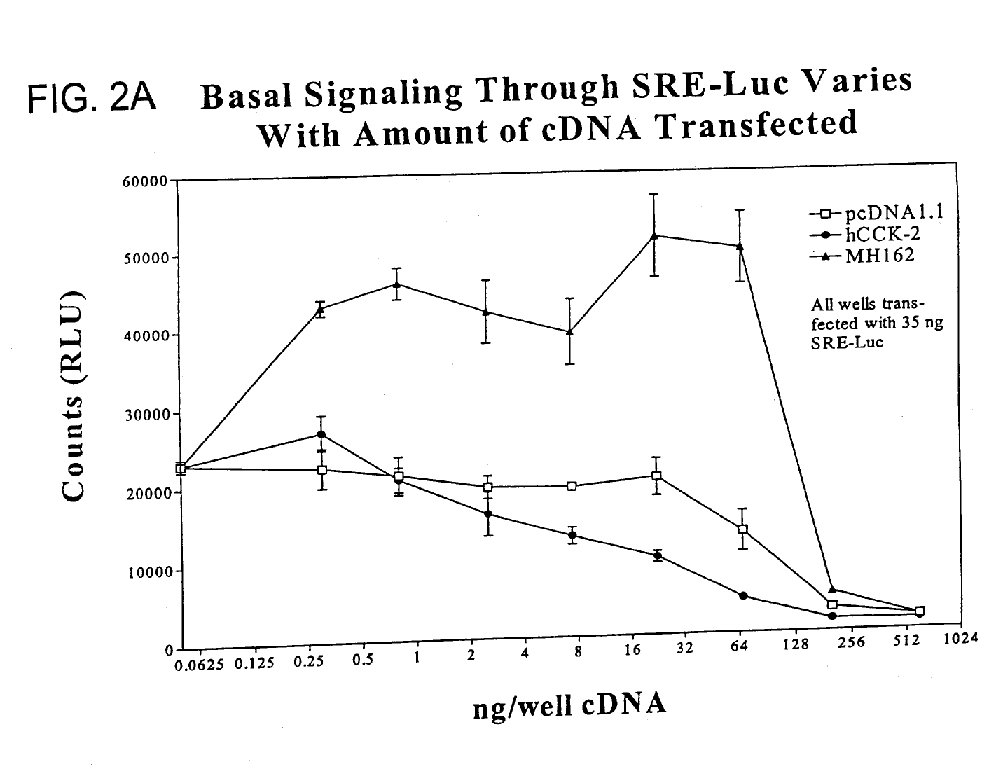

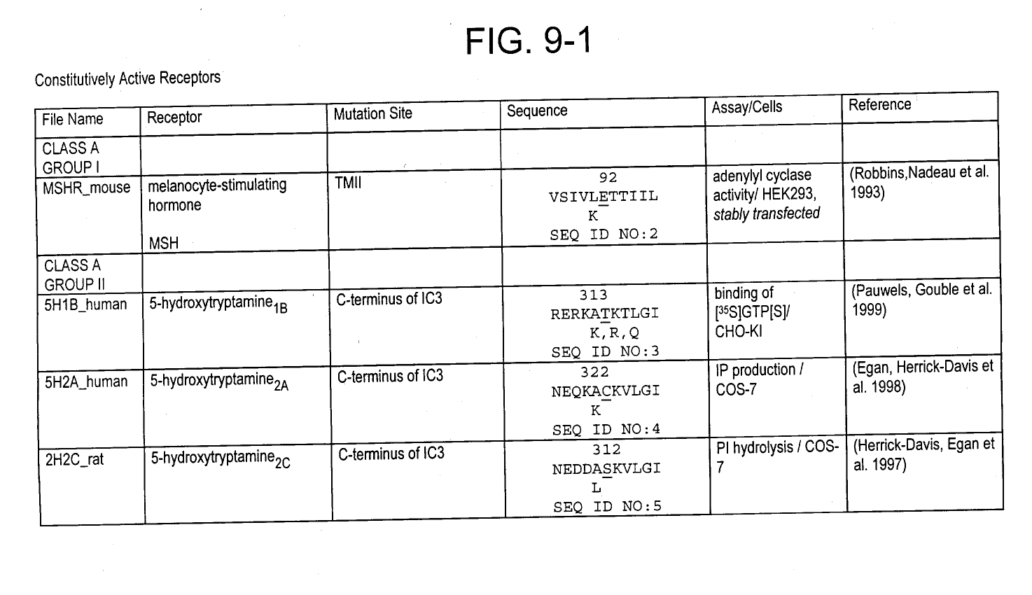

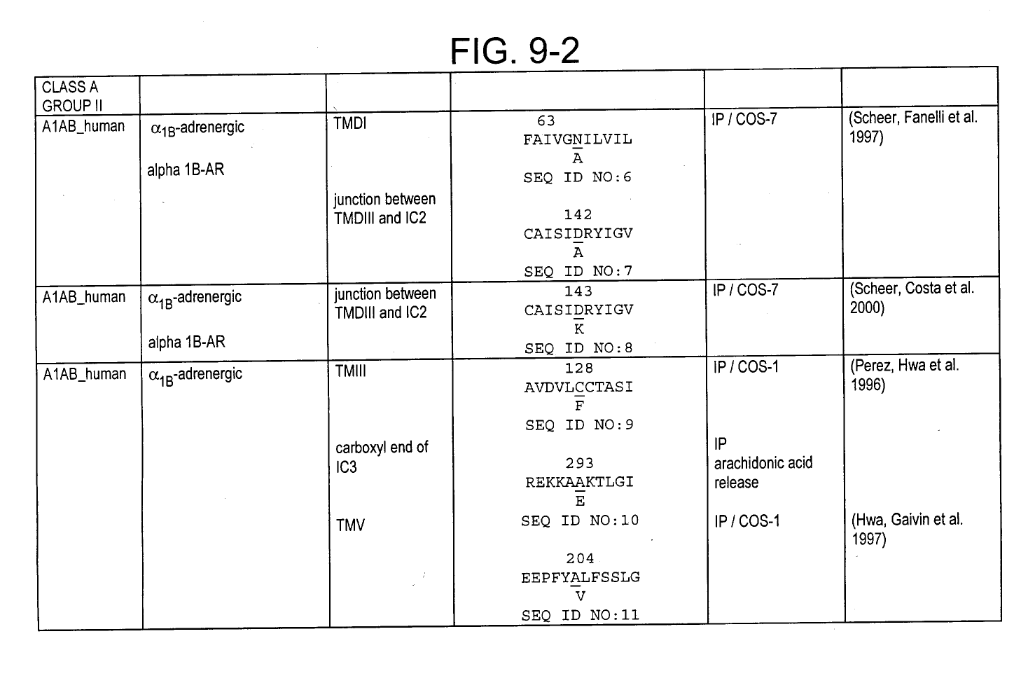

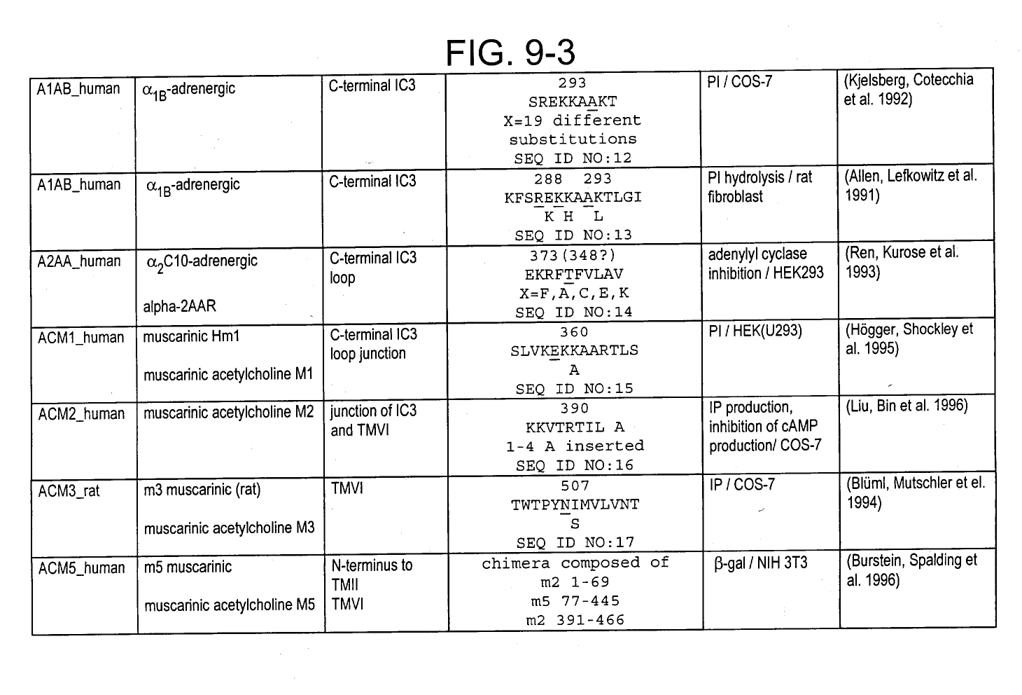

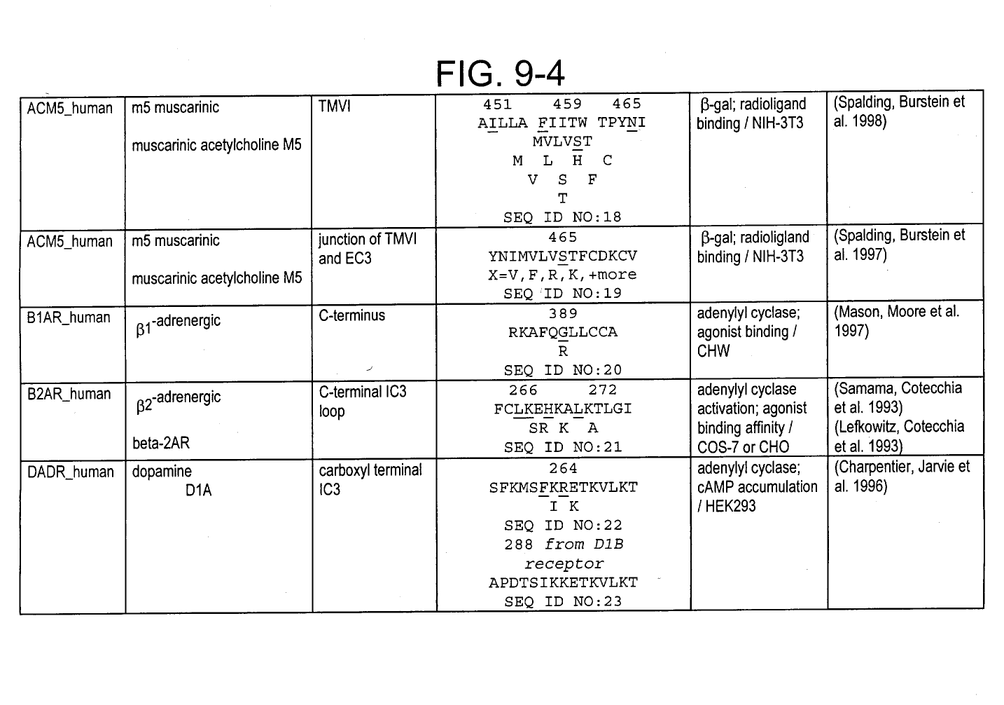

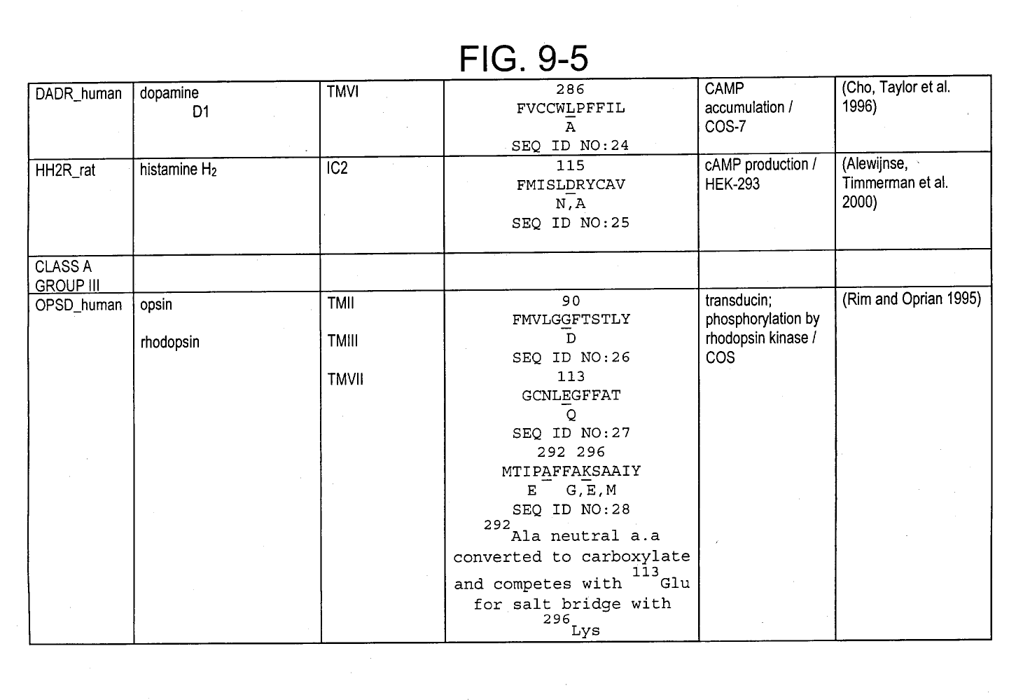

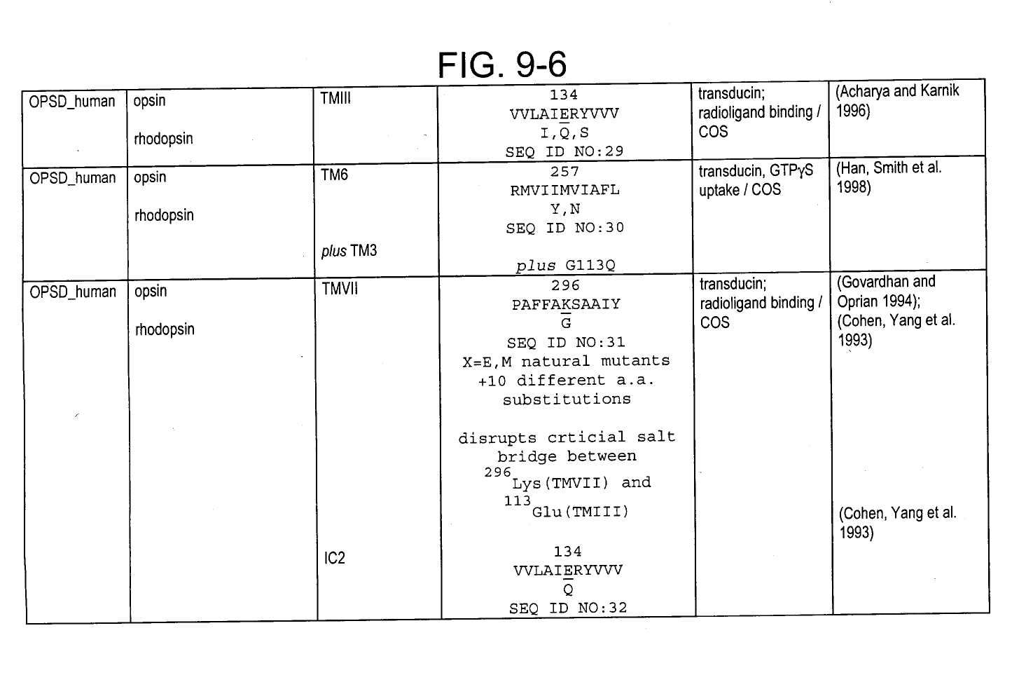

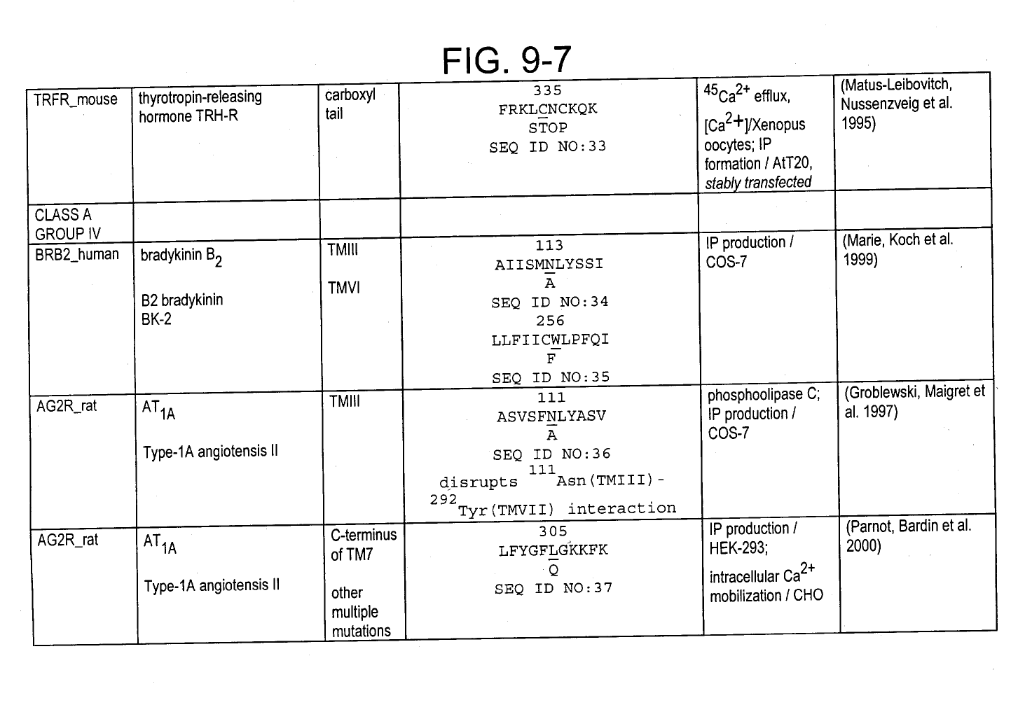

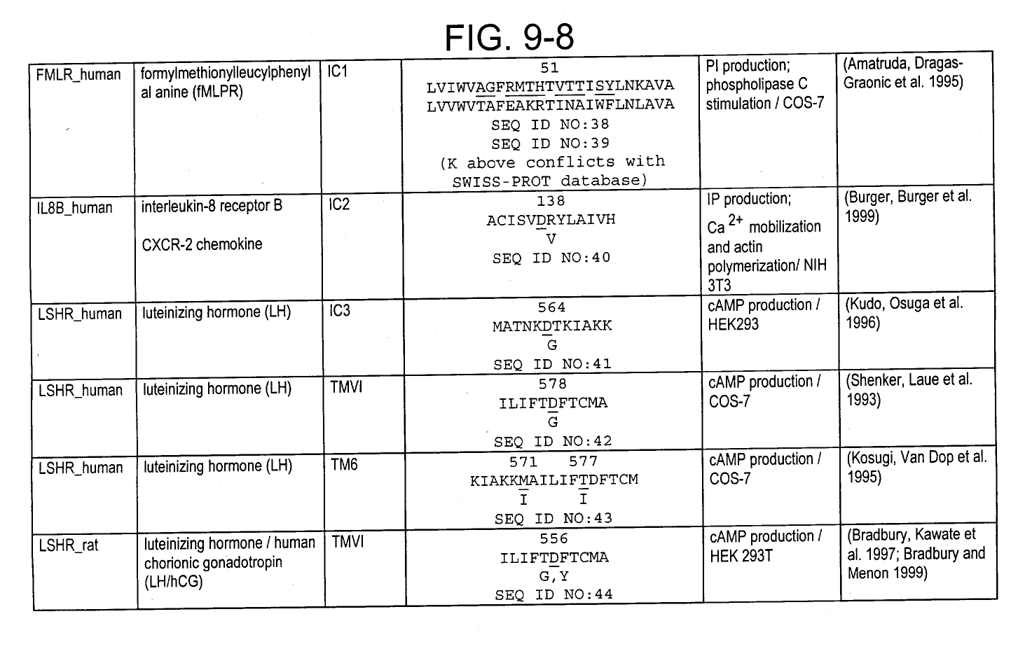

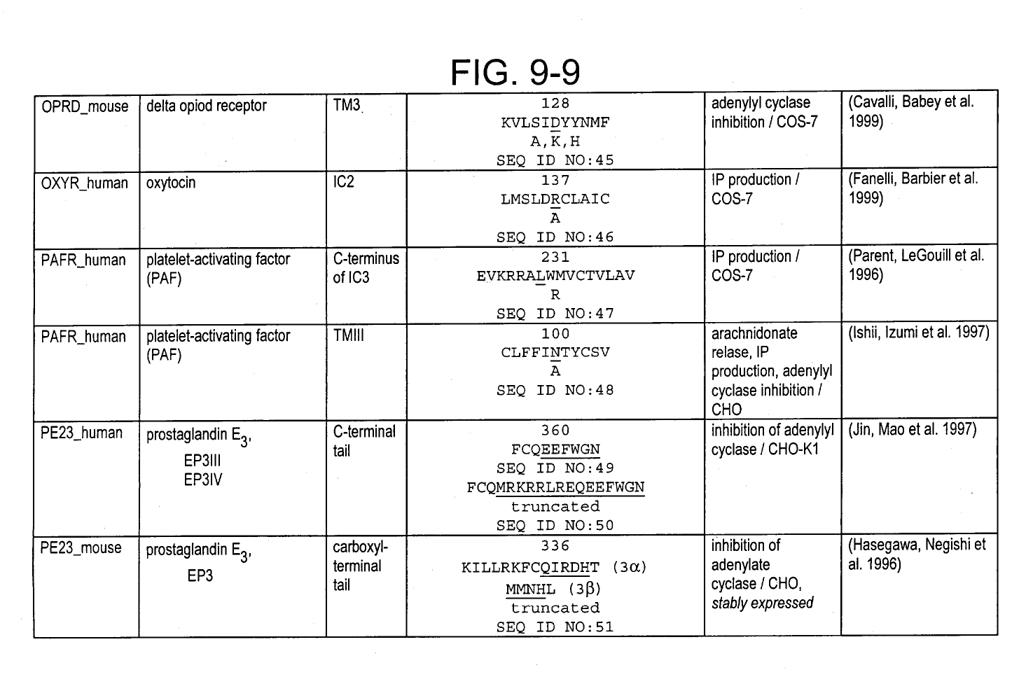

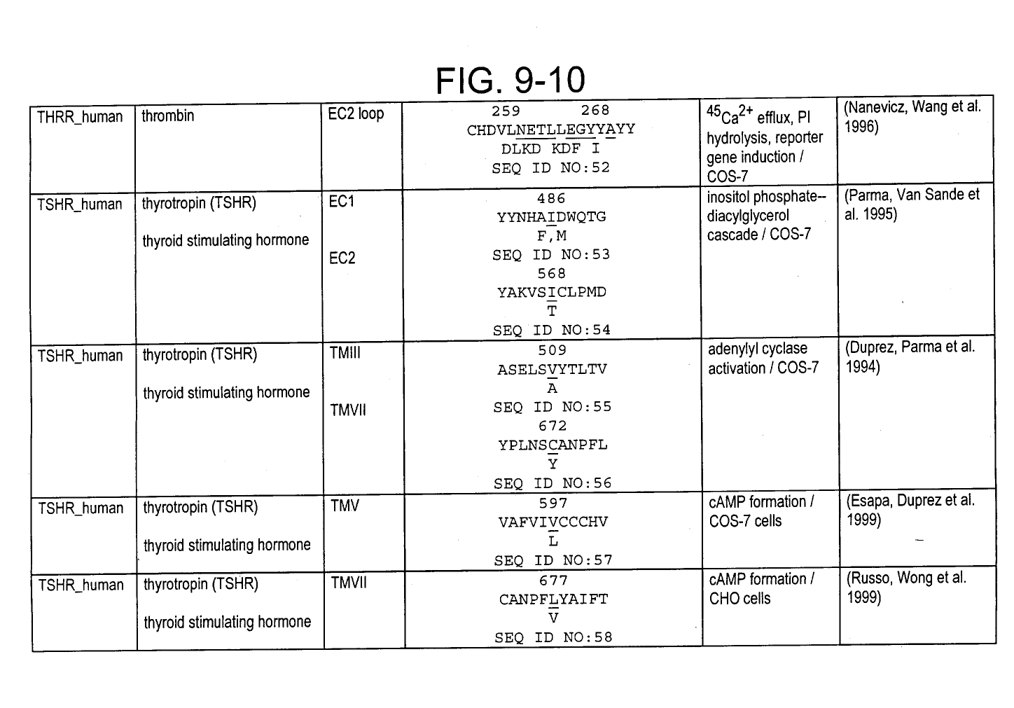

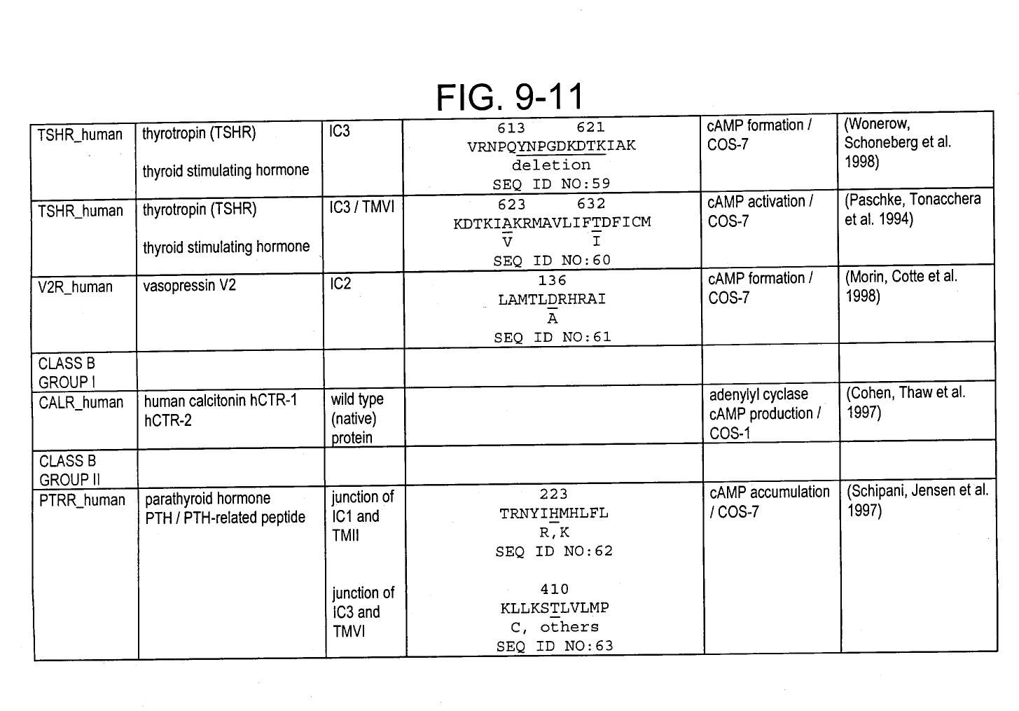

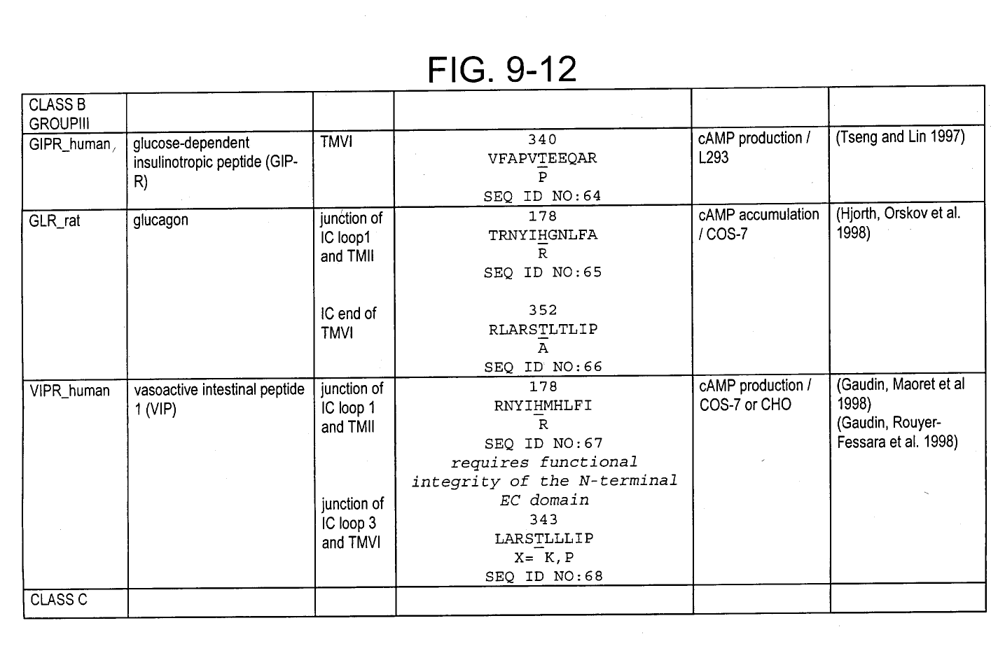

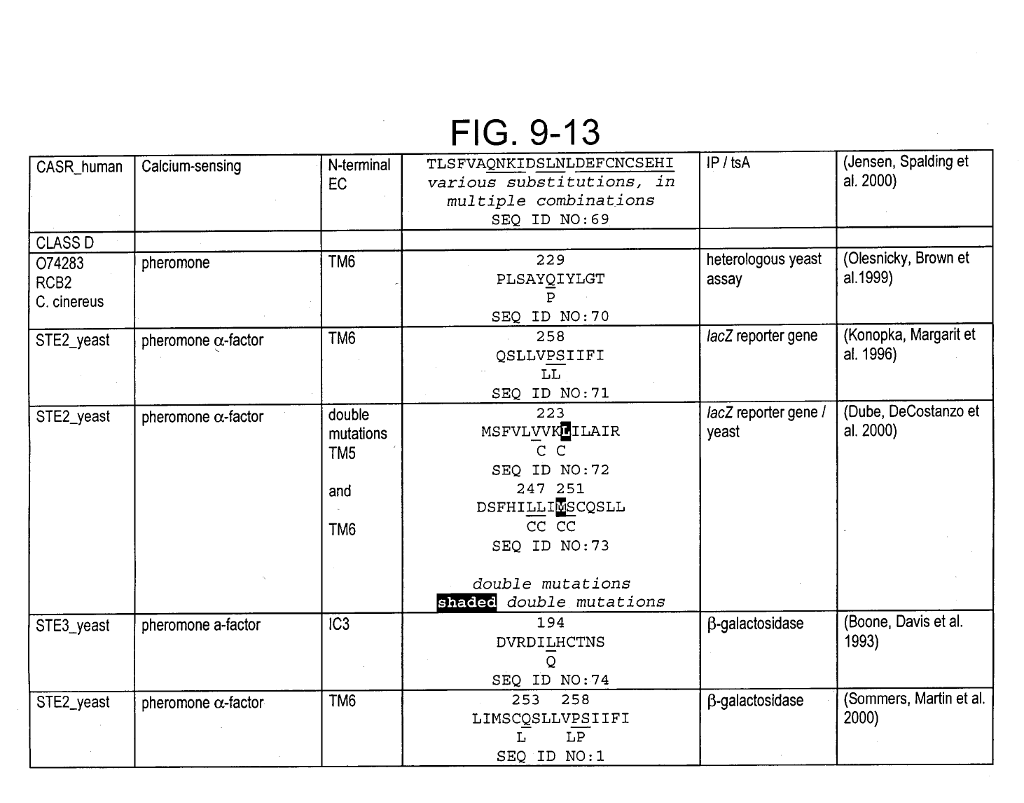

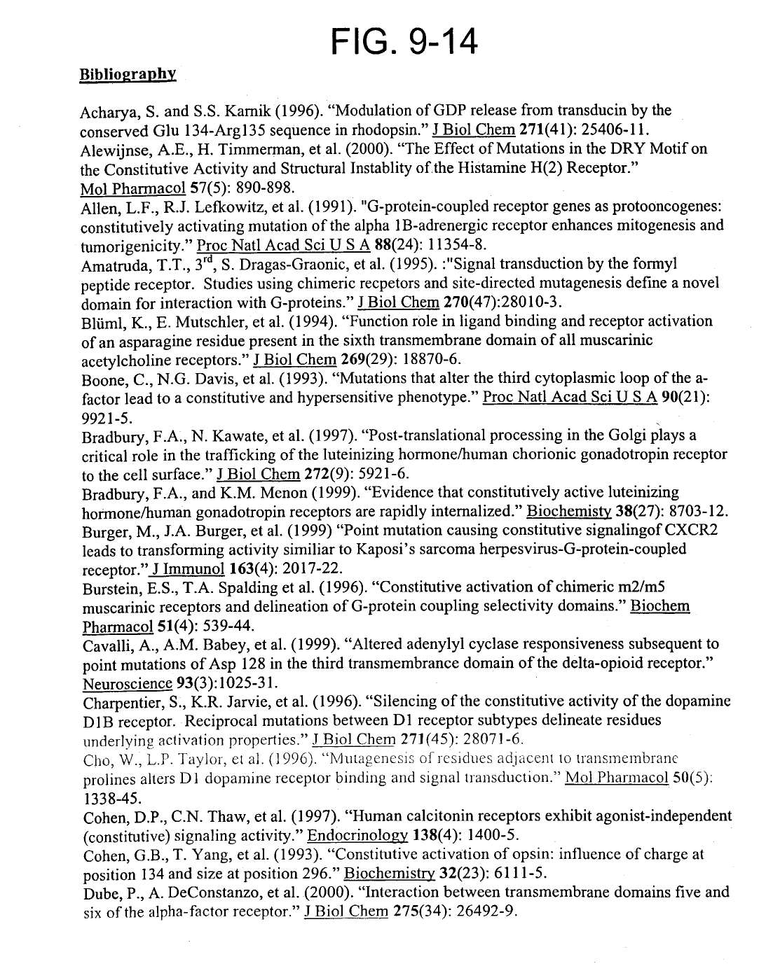

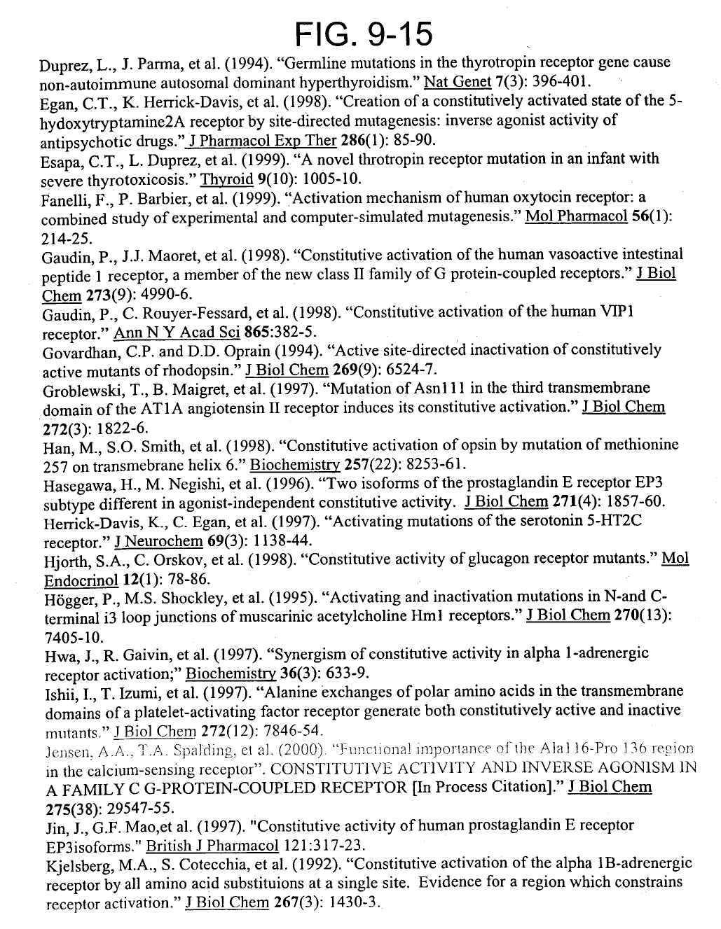

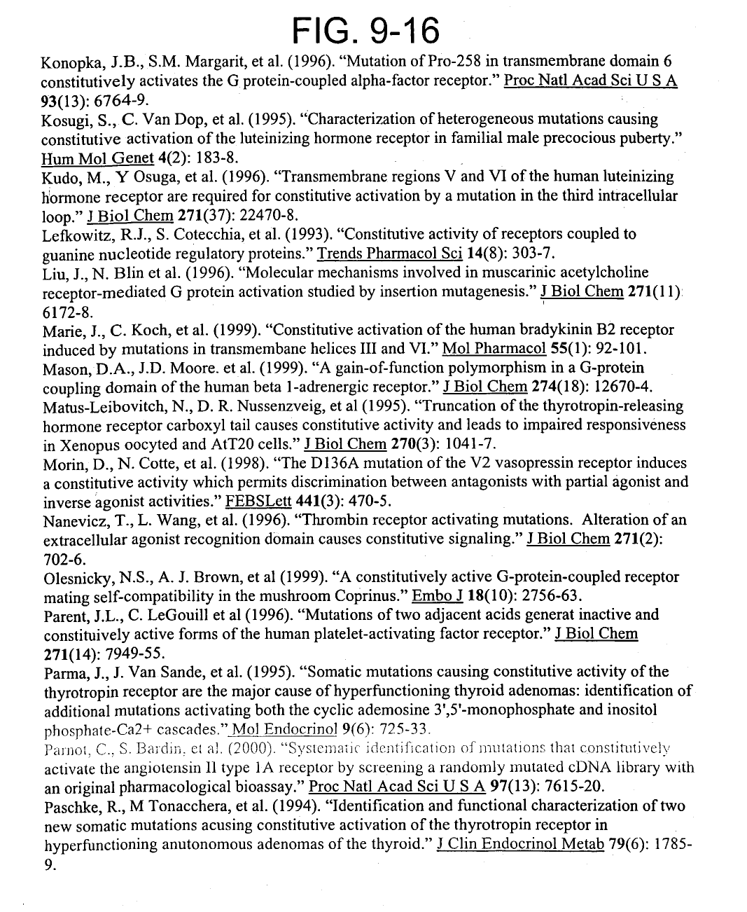

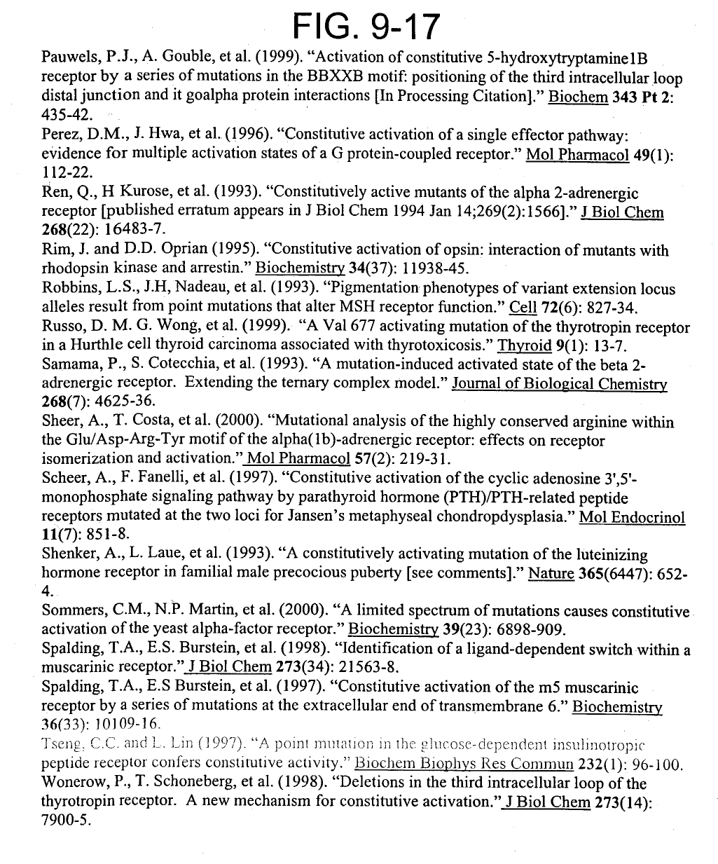

A constitutively active receptor is a receptor with a higher basal activity level than the corresponding wild-type receptor. A constitutively active receptor is also a receptor possessing the ability to spontaneously signal in the absence of activation by a positive agonist. This term includes wild-type receptors that are naturally constitutively active (e.g., naturally occurring receptors, including naturally occurring polymorphic receptors). Constitutively active receptors include constitutively active G protein-coupled receptors (e.g., opiate receptors), single transmembrane domain receptors (e.g., the erythropoietin receptor (EPO receptor)), and nuclear receptors (e.g., steroid hormone receptors, such as the estrogen receptor). Examples of known constitutively active receptors are shown in FIG. 9 herein and in FIG. 1 of Juppner et al., Curr. Opin. Nephrol, Hypertens. 3:371-378, 1994.

A hypersensitive receptor is a receptor having the ability to amplify the input of a ligand, as compared to the corresponding wild type receptor. Accordingly, such receptors deliver an increased receptor-induced signal in response to a ligand compared to a corresponding negative control receptor, which may occur either in terms of increased potency (i.e., increased response relative to the negative control receptor at a given concentration of a ligand or drug) or increased efficacy (i.e., increased maximal ligand stimulation), or both. The increased ligand induced signal of hypersensitive receptors may be apparent at ligand concentrations which induce maximal or sub-maximal ligand stimulation, or both.

A hyposensitive receptor is a receptor having the ability to reduce the response to a ligand, as compared to the corresponding wild type receptor. Hyposensitive receptors deliver a decreased receptor-induced signal in response to a ligand compared to a corresponding negative control receptor either in terms of decreased potency (i.e., decreased response relative to the negative control receptor at a given concentration of a ligand or drug) or decreased efficacy (i.e., decreased maximal ligand stimulation), or both. The decreased ligand induced signal of hyposensitive receptors may be apparent at ligand concentrations which induce maximal or sub-maximal ligand stimulation, or both.

A silenced receptor is a receptor having a decreased level of basal activity compared to the corresponding wild type receptor. As a second, non-obligatory criterion, a silenced receptor may also not transmit a signal or transmit a reduced signal in response to ligand binding. A fully silenced receptor has little or no activity, whereas a partially silenced receptor has reduced basal activity compared to the corresponding wild type receptor.

A non-functional receptor is a receptor that neither signals in the absence of ligand nor in response to ligand binding. A non-functional receptor could also be a receptor that does not bind ligand, and therefore does not transmit a signal in response to ligand binding. According to the invention, any mutation that eliminates signaling of a receptor qualifies as a non-functional receptor.

A naturally-occurring receptor refers to a form or sequence of a receptor as it exists in an animal, or to a form of the receptor that is homologous to the sequence known to those skilled in the art as the “wild-type” sequence. Those skilled in the art will understand wild type receptor to refer to the conventionally accepted wild-type amino acid consensus sequence of the receptor, or to a naturally-occurring receptor with normal physiological patterns of ligand binding and signaling. A mutant receptor is a form of the receptor in which one or more amino acid residues in the predominant receptor occurring in nature, e.g., a naturally-occurring wild-type receptor, have been either deleted, inserted, or replaced. Mutant receptors may be generated by identifying regions of homology between a receptor that is not considered to have altered signaling and one or more receptors having altered signaling and introducing mutations, using standard techniques, into the identified homologous regions, for example, the regions identified in the database shown in FIG. 9, or in Juppner, supra.

Chimeric G Proteins

The present invention provides use of specific response elements that are sensitive to signaling through each of Gq, Gs, Gi, and Go. For example, the CCK-2 receptor signals through Gq, the MC-4 and PTH receptors signal through Gs, and the mu opioid receptor signals through Gi coupling. Traditionally, Gi coupling has been detected using the cAMP-response element (CRE), which is sensitive to Gαi mediated changes in intracellular levels of cAMP. Signaling through the rat mu opioid receptor via Gαi inhibits adenylate cyclase, causing a decrease in intracellular cAMP. Therefore, an increase in rat mu opioid receptor signaling induces a decrease in CRE mediated reporter activity.

This traditional method of detecting Gi (and Go) coupling has several disadvantages. First, detecting Gαi-mediated inhibition of cAMP requires induction of simultaneous positive effects, e.g., by forskolin on adenylate cyclase, and these positive effects need to be overcome by Gαi mediated signaling. In addition, since the simultaneous stimulatory effects are typically induced by a mechanism that uniformly acts on all cells in the assay (e.g., forskolin-stimulated cAMP production), the detection of a ligand-stimulated decrease in intracellular cAMP relies on whether a large enough percentage of the cells are successfully transfected with, and express, the Gαi-coupled receptor molecule. Moreover, when using transient transfection assays, instead of stably transfected cell lines, inter-experimental variation occurs because the percentage of cells transfected from one experiment to the next is difficult to control.

A positive assay for Gi and Go coupling (i.e., an assay that yields an increase in luciferase activity upon receptor activation, instead of a negative assay that yields a decrease in luciferase activity upon receptor activation), provides a more detectable output signal and less inter-assay variation. Gi or Go coupling can be detected by altering the signaling pathway generated by Gi or Go coupled receptors. For example, a chimeric G protein (Gq5i), Broach and Thorner, Nature 384 (Suppl.): 14-16 (1996), that contains the entire Gαxq protein having the five C-terminal amino acids from Gαi attached to the C-terminus of Gαq has been generated. This chimeric G protein is recognized as Gαi by Gαi coupled receptors, but switches the receptor induced signaling from Gαi to Gαq. This allows Gαi receptor coupling to be detected using a positive assay by use of the Gαq responsive SMS-Luc or SRE-Luc construct (Stratagene, La Jolla, Calif.). SMS and SRE preferably respond to Gαq mediated inositol phosphate and calcium production. It is of note that detection can be carried out in the absence of forskolin pre-stimulation of cells.

Other chimeric G proteins that can be used according to the methods of the invention include those shown in Appendix 1 (G Protein Users Manual, http://gweb1.ucsf.edu/labs/Conklin/technical/GproteinManual.html) and described in Milligan, G. and S. Rees, TIPS 20:118-124, 1999, and Conklin et al., Nature 363: 274-276, 1993, incorporated by reference herein. Moreover, any other chimeric G protein can be constructed by replacing or adding at least 3 amino acids, usually at least 5 amino acids, from the carboxyl terminus of a G protein (e.g., Gi, Gq, Gs, Gz, or Go) to a second G protein (e.g., Gi, Gq, Gs, Gz, or Go) which is either full-length or includes at least 50% of the amino terminal amino acids.

Generally, the carboxyl-terminus of the G alpha protein subunit is a key determinant of receptor specificity. For example, the Gq alpha subunit (alpha q) can be made to respond to Gi alpha-coupled receptors by replacing its carboxyl-terminus with the corresponding Gi2 alpha, Go alpha, or Gz alpha residues. In addition, C-terminal mutations of Gq alpha/Gi alpha chimeras show that the critical amino acids are in the−3 and−4 positions, and exchange of carboxyl-termini between Gq alpha and Gs alpha allows activation by receptors appropriate to the C-terminal residues. Furthermore, replacement of the five carboxyl-terminal amino acids of Gq alpha with the Gs alpha sequence permitted a certain Gs alpha-coupled receptor (the V2 vasopressin receptor, but not the beta 2-adrenoceptor) to stimulate phospholipase C. Replacement of the five carboxyl-terminal amino acids of Gs alpha with residues of Gq alpha permitted certain Gq alpha-coupled receptors (bombesin and V1a vasopressin receptors, but not the Oxytocin receptor) to stimulate adenylyl cyclase. Thus, the relative importance of the G alpha carboxyl-terminus for permitting coupling to a new receptor depends on the receptor with which it is paired.

Any other G protein chimera that is capable of switching the signaling from one G-protein coupled receptor to another pathway can also be used according to the invention.

Receptor Assays

The present invention provides methods of identifying constitutively active, hypersensitive, hyposensitive, silenced, or non-functional receptors. Accordingly, the invention provides a reporter assay system, i.e., any combination of vectors typically used for measuring transcriptional activation, to identify constitutively active, hypersensitive, hyposensitive, silenced, or non-functional receptors. A typical reporter assay system includes at least a reporter construct and an expression vector encoding the polypeptide that activates (e.g., directly) or causes to activate (e.g., indirectly) expression of the reporter construct. The reporter assay system may also include additional expression vectors encoding other polypeptides that participate in activation of the reporter construct. In a reporter assay system, a response element responsive to signaling through a particular receptor is attached to a reporter gene in combination with a transcriptional promoter.

The invention features a reporter assay system in which a response element, responsive to signaling through a particular receptor, is attached to a reporter gene in combination with a transcriptional promoter. More specifically, the expression of the reporter gene is controlled by the activity of the chosen receptor. This method involves the steps of (1) identifying a response element that is sensitive to signaling by a specific receptor polypeptide (e.g., by eliciting an increase or decrease in gene expression upon receptor activation); (2) operably linking the response element and a promoter (if the promoter is not included in the response element) to a reporter gene; and (3) comparing the basal level reporter activity of a putative receptor with altered signaling to a negative control by generating dose response curves, where an increase or decrease in basal level reporter activity compared to the negative control over a range of at least two concentrations, identifies a constitutively active receptor or silenced receptor, respectively. Similarly, an increase or decrease in ligand stimulated activity compared to the negative control over a range of at least two concentrations indicates the identification of a hypersensitive or hyposensitive receptor, respectively, and an absence of ligand-stimulated activity, compared to a corresponding functional receptor, indicates the identification of a nonfunctional receptor. It is important to note that hypersensitive receptors may not necessarily have any detectable increase in basal activity. An important aspect of the method is the generation of dose response curves. While a range of two concentrations is acceptable, a range of three, five, or greater than ten concentrations allows for greater reliability and reproducibility. The concentrations can span two or greater logarithmic intervals. The invention also provides a reporter assay system capable of identifying a G protein coupled receptor with altered signaling by using a chimeric G protein to elicit a positive signal.

The methods of the invention are used to screen for receptors exhibiting constitutive, hypersensitive, hyposensitive, silenced, or non-functional activity. The receptor can be any receptor identified as a candidate constitutively active, hypersensitive, hyposensitive, or non-functional receptor. In addition, the response element can be any response element that is sensitive to signaling through the identified candidate constitutively active receptor. For example, in reporter assays for identifying constitutively active receptors that are coupled to different G proteins, one would select response elements that are sensitive to signaling through receptors coupled to G proteins. In particular examples, the somatostatin promoter (which has included a number of different response elements) (SMS) is activated by coupling of receptors to either Gαq or Gαs; the serum response element (SRE) is activated by receptor coupling to Gαq; the cAMP response element (CRE) is activated by receptor coupling to Gαs and inhibited by coupling to Gαi; and the TPA response element (sensitive to phorbol esters) is activated by receptor coupling to Gαq. Each of these response elements can be employed in a reporter assay to generate a readout for the basal level activity of a specific G protein-coupled receptor.

In addition, a reporter construct for detecting receptor signaling might include a response element that is a promoter sensitive to signaling through a particular receptor. For example, the promoters of genes encoding epidermal growth factor, gastrin, or fos can be operably linked to a reporter gene for detection of G protein-coupled receptor signaling. Another example includes the TPA response element, which is sensitive to phorbol ester induction. It will be appreciated that a wide variety of reporter constructs can be generated that are sensitive to any of a variety of signaling pathways induced by signaling through a particular receptor (e.g., a second messenger signaling pathway). Accordingly, the methods of the invention may be used to identify other types of constitutively active receptors, including receptors that are single transmembrane receptors or nuclear receptors, by simply selecting a response element that is sensitive to the particular receptor and positioning the response element upstream of a reporter gene in a reporter construct. For example, the elements AP-1, NF-κb, SRF, MAP kinase, p53, c-jun, TARE can all be positioned upstream of a reporter gene to obtain reporter gene expression. Additional response elements, including promoter elements, can be found in the Stratagene catalog (PathDetect® in Vivo Signal Transduction Pathway cis-Reporting Systems Introduction Manual or PathDetect® in Vivo Signal Transduction Pathway trans-Reporting Systems Introduction Manual, Stratagene, La Jolla, Calif.).

The constitutive activity, hypersensitivity, hyposensitivity, silencing, or lack of activity, respectively, of a particular receptor can also be measured by any assay typically used to measure the basal and/or ligand-stimulated activity of the receptor. For example, changes in basal level second messenger signaling may be assessed to identify constitutively active receptors, including, but not limited to changes in basal levels of cAMP, cGMP, ppGpp, inositol phosphate, or calcium ions.

As noted above, some receptors (e.g., some wild-type receptors) are naturally constitutively active. Such naturally occurring constitutively active receptors are identified by simply comparing the basal activity of the wild-type receptor to that of a negative control. A suitable negative control is, for example, a cell lacking expression of the natural wild-type receptor (e.g., a cell transfected with an empty expression vector, or a cell transfected with a different receptor that has been previously established to lack constitutive activity (preferably both an empty expression vector and a non-constitutively active reference receptor are used)).

Alternatively, mutant receptors having constitutive activity can be identified by comparing the basal level of signaling of the mutant constitutively active receptor to the basal level of signaling of the wild-type receptor. The constitutive activity of a mutant or naturally occurring receptor may also be established by comparing the basal level of signaling, such as second messenger signaling, of the receptor to the basal level of signaling of the corresponding wild-type receptor. Any assay typically used for measuring the ligand-stimulated activity of the wild-type receptor may also be used to measure the basal level activity of a mutant receptor. It is common for a constitutively active receptor, e.g., a polymorphic constitutively active receptor, that is associated with a disease phenotype, to display a relatively small increase in constitutive activity (e.g., as little as a 25% increase). The basal activity of a constitutively active receptor can be confirmed by its decrease in the presence of an inverse agonist.

These simple principles can easily be applied to identify a wide range of constitutively active G protein-coupled receptors. As but one example, ligand-dependent activation of the melanocortin-4 (MC-4) receptor is assayed by measuring an increase in cAMP production (Huszar et al., Cell 88:131-141, (1997)). Additional examples of G protein-coupled receptors having intracellular second messenger signaling pathways that may be evaluated to identify constitutively active forms of receptors include the GLP-1 receptor (adenylate cyclase and phospholipase C (PLC)) and the parathyroid hormone receptor (PTH) (see Dillon et al., Endocrinology 133(4):1907-1910, (1993); Whitfield and Morley, TiPS, 16:382-385, 1995). Other G protein-coupled receptors bind to certain intracellular molecules in their activated states. For example, the mu opioid receptor induces an increased level of GTP binding by receptor-activated G protein (Gαi) (see, e.g., Befort et al., J. Biol. Chem. 274(26):18574-18581, (1999)).

The activity of other types of receptors (e.g., non-G protein-coupled receptors such as single transmembrane domain receptors and nuclear receptors) can also be measured via the biochemical pathway they induce. For example, binding of the ligand EPO to the EPO receptor activates the JAK2-STAT5 signaling pathway (see, e.g., Yoshimura et al., Curr. Opin. Hematol., 5(3): 171-176, 1998).

The basic principles that apply to the identification of receptors having increased basal level activity (constitutively active receptors) are directly applicable to the identification of receptors having reduced basal level activity (e.g., silenced receptors) and also to receptors that are hypersensitive or hyposensitive. Receptors that are hypersensitive or hyposensitive are identified by comparing the ligand-induced activity of the wild-type receptor to the ligand-induced activity of the mutant or polymorphic receptor, a hypersensitive or hyposensitive receptor being identified by its ability to display a stronger or weaker signal, respectively, to a given concentration of ligand than the wild-type receptor. A hypersensitive or hyposensitive receptor may therefore be characterized in that it exhibits an increased or decreased response, respectively, to a specific concentration of ligand, compared to the response of a wild-type receptor to the same concentration of ligand. For example, if 5 μM ligand induces a 5-fold stimulation of activity in a wild-type receptor, compared to a negative control, 5 μM ligand may stimulate a 10-fold stimulation in activity in a hypersensitive receptor, compared to the same negative control. Candidate hypersensitive receptors can thus be stimulated with a low concentration of ligand (below saturating levels of ligand) and the receptor induced signal measured. An increase in ligand-stimulated activity compared to the wild-type receptor indicates the identification of a hypersensitive receptor. Similarly, if 5 μM ligand induces a 5-fold stimulation of activity in a wild-type receptor, compared to a negative control, 5 μM ligand may stimulate a 2-fold stimulation in activity in a hyposensitive receptor, compared to the same negative control.

Non-functional receptors can be generated using techniques similar to those for identifying hypersensitive receptors, and tested for an absence of ligand stimulated response compared to the functional wild-type receptor.

The examples described herein illustrate the sensitivity of reporter gene constructs in detecting mutation or polymorphism induced alterations in the basal level of receptor mediated second messenger signaling. The sensitivity of the assay is markedly enhanced by profiling mutation or polymorphism induced alteration of activity over a concentration range of transfected receptor cDNAs; this is done while holding the concentration of reporter gene (and in some cases chimeric G-protein) constant. Alternatively and additionally, dose response curves of the transfected receptor cDNAs can also be carried out at different defined doses of reporter gene co-transfections to further enhance the sensitivity of the assay. Over the majority of the curve, wild type and functionally altered mutant/polymorphic receptors can be differentiated. The importance of generating a curve is highlighted at the high and low concentrations of transfected receptor cDNA, where functional activity of the mutants may overlap with wild type. The examples therefore both illustrate that receptors with altered signaling can be reliably and reproducibly identified by generating dose response curves and demonstrate that experimental artifacts may occur in traditional receptor assays that do not include assessment of signaling over a dose range. These artifacts may mask the activity of a receptor with altered signaling relative to a negative control or a wild type receptor.

Applications

Once identified, receptors having altered signaling may be used in drug screening assays, for example, large scale high throughput screening assays, to identify ligands (e.g., including peptide, non-peptide, and small molecule ligands). These ligands may, upon further experimentation, prove to be valuable therapeutic drugs for treatment of a disease or disorder for which activation or inhibition of the receptor (by, e.g., an agonist, inverse agonist, or antagonist, respectively) has a beneficial therapeutic effect.

For example, ligands (e.g., a hormone or a drug) that bind a particular constitutively active receptor may be identified using a reporter assay system as described herein, in which the cells are contacted with a ligand and assayed for ligand-dependent activation or inhibition of the reporter construct, an increase or decrease in the ligand-dependent activation, compared to ligand-independent signaling, indicating the presence of an agonist or antagonist, respectively. Ligands that activate or inhibit a particular receptor by increasing or decreasing receptor activity may, upon further experimentation, prove to be valuable therapeutic drugs for treatment of disease.

Alternatively, the assay systems of the present invention may be used to screen for genetic polymorphisms or mutations that alter (i.e., increase or decrease) the basal or ligand-stimulated signal generated by a particular receptor. Thus, the receptors of the present invention can also be used to identify the underlying mechanism by which a genetic polymorphism or mutation contributes to a particular disease or disorder or enhances health. For example, the identified polymorphisms or mutations can result in agonist independent signaling, particularly agonist independent signaling that causes disease. Furthermore, the identified polymorphisms or mutations can result in an altered response to a drug. The assay systems of the present invention can also be used to detect mutation-induced sensitivity of a receptor to ligand induced signaling (e.g., by identifying a hypersensitive receptor). With the emergence of pharmacogenomics, rapid methods of screening for functionally important polymorphisms or mutations are highly valuable.

When applied to orphan receptors (wild-type or mutant), the methods of the invention in conjunction with a panel of reporter gene constructs that are sensitive to different signaling pathways (e.g., SRE-Luc, SMS-Luc, and CRE-Luc) can be used to predict the second messenger pathway that will be activated by the endogenous receptor ligand (e.g., cAMP, inositol phosphate production). This information will facilitate and accelerate both the identification of cognate endogenous ligands (i.e., the de-orphaning of a receptor), and the discovery of drugs that act on orphan receptors by the use of the inventive high-throughput screening based techniques. This allows drug screening efforts to be more focused and to be carried out at reduced cost. In addition, no knowledge of the endogenous ligand is needed as a prerequisite for drug screening (which is a prerequisite of competitive binding assays).

The following examples are provided for the purpose of illustrating the invention and should not be construed as limiting.

### EXAMPLE 1

Constitutively Active CCK-2 Receptor

Wild type CCK-2 receptor (Gq coupled) and a constitutively active mutant (MH162) were assessed over a wide range of DNA co-transfection amounts. DNA “dose response” curves were used to demonstrate constitutive activity independent of ligand stimulation. Wells were co-transfected with varying concentrations (i.e. 5 ng DNA/well, 35 ng DNA/well, and 150 ng DNA/well) of the SRE-luciferase reporter construct. Cells were assayed the following day using the LucLite Luciferase Assay Kit (Packard).

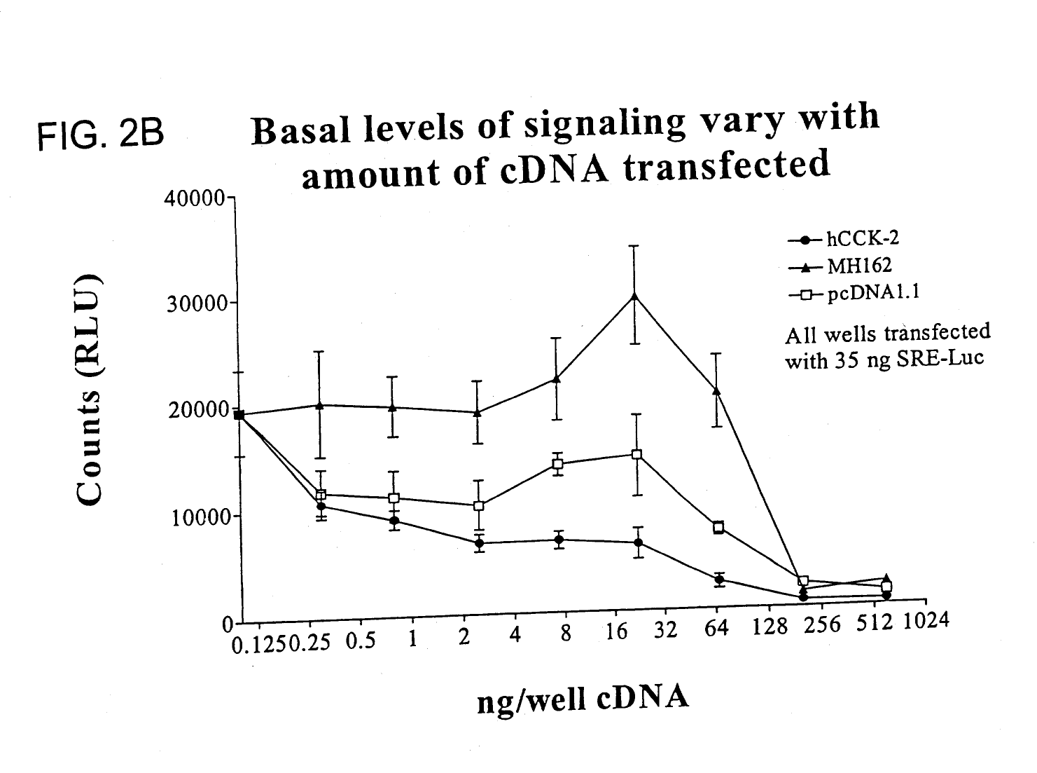

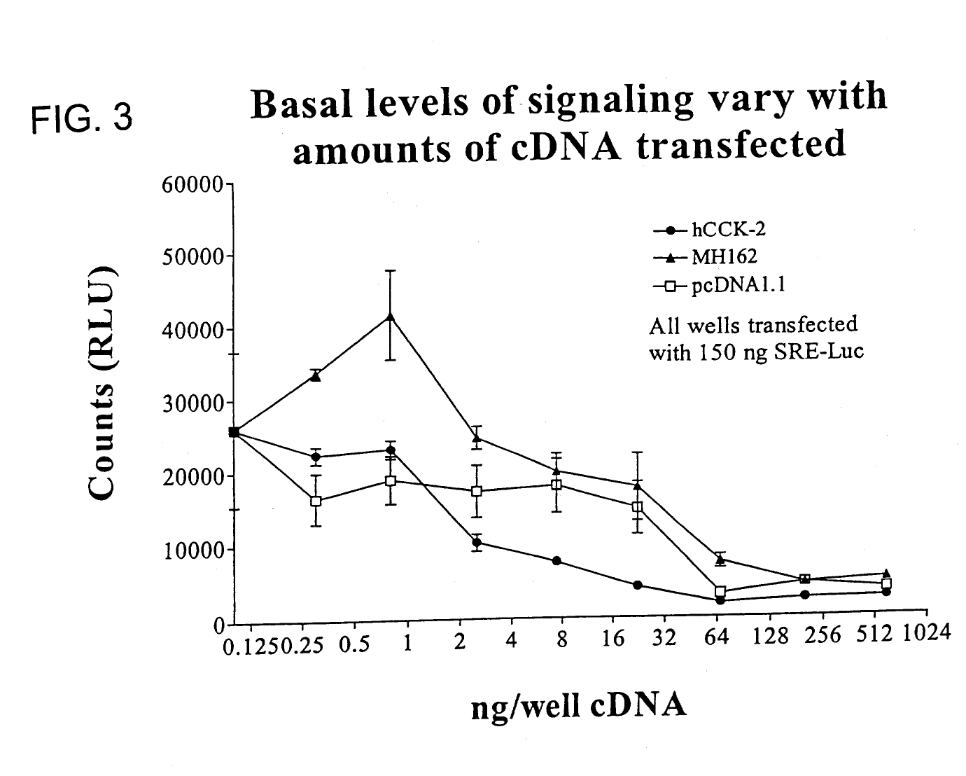

For each of the illustrated concentrations of co-transfected SRE-luciferase constructs, the assay successfully distinguished wild type from constitutively active receptors over specific ranges of transfected receptor cDNA/well (FIGS. 1-3). Wild type basal (unstimulated) signaling was less than or approximated signaling in cells transfected with the empty expression vector, pcDNA 1.1. In contrast, when the cDNA encoding the constitutively active mutant was transfected over a wide concentration range (FIGS. 1-3), signaling was induced which significantly exceeded both the wild type value and that observed with the empty expression vector.

### EXAMPLE 2

Constitutively Active MC-4 Receptor

Wild type MC-4 (Gs coupled) and a mutant MC-4 receptor (MC4-M12) were assessed over a wide range of DNA co-transfection amounts. DNA “dose response” curves were used to demonstrate constitutive activity independent of ligand stimulation. Each well was co-transfected with 35 ng reporter overnight.

Cells were assayed the following day using the LucLite Luciferase Assay Kit (Packard).

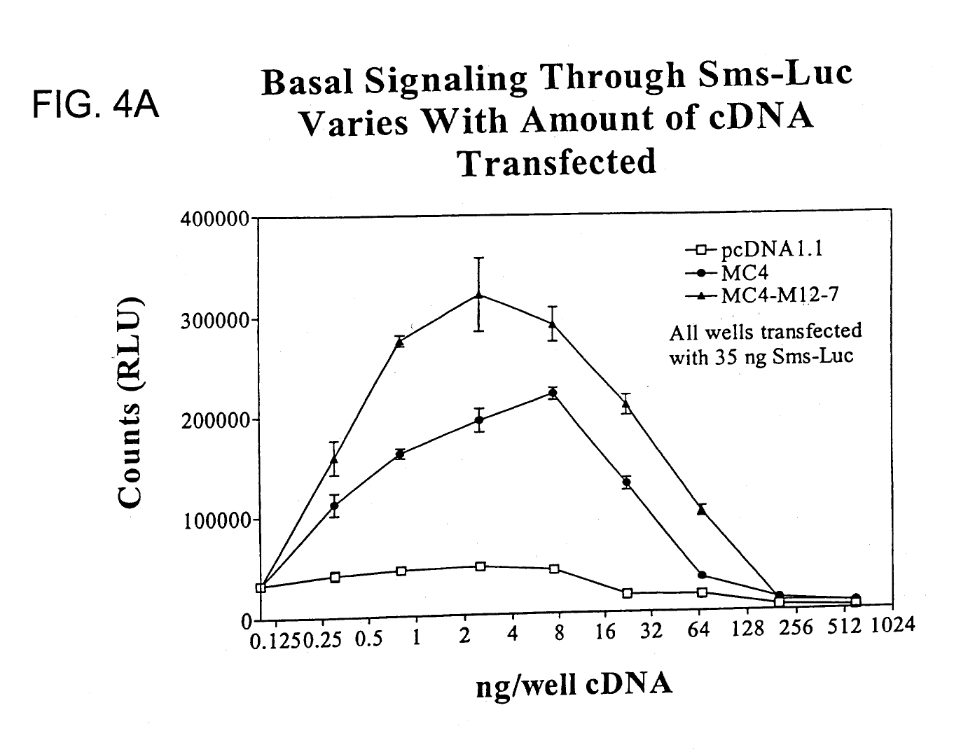

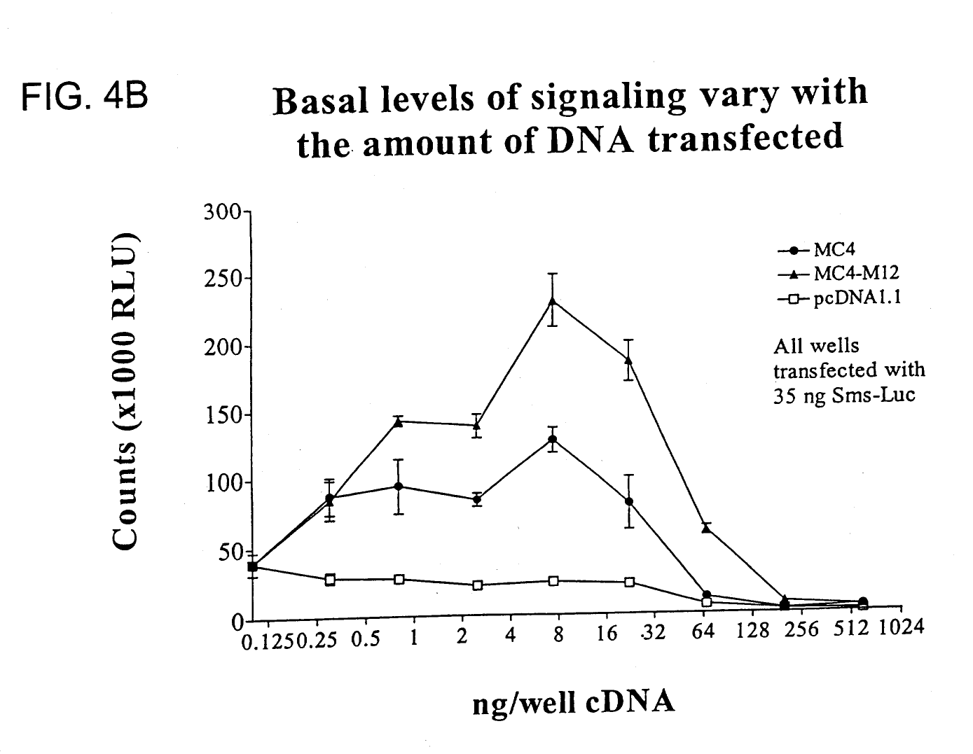

FIGS. 4A-B contrast the wild type MC-4 receptor (Gs coupled) with a receptor mutant which is more constitutively active (MC4-M12). Over a wide range of transfected cDNA (see figure), the basal level of signaling of the wild type receptor is elevated compared to the “empty” expression vector pcDNA1.1; therefore the wild type receptor is constitutively active. A further increase in basal signaling is observed with expression of the cDNA encoding the MC-4 receptor with an activating point mutation (MC4-M12).

### EXAMPLE 3

Constitutively Active PTH Receptor

The wild type parathyroid hormone (PTH) receptor (Gs coupled) and two constitutively active PTH receptor mutants (H223R and T410P) were assessed over a wide range of DNA co-transfection amounts. DNA “dose response” curves were used to demonstrate constitutive activity independent of ligand stimulation. Each well was co-transfected with 35 ng reporter overnight. Cells were assayed the following day using the LucLite Luciferase Assay Kit (Packard).

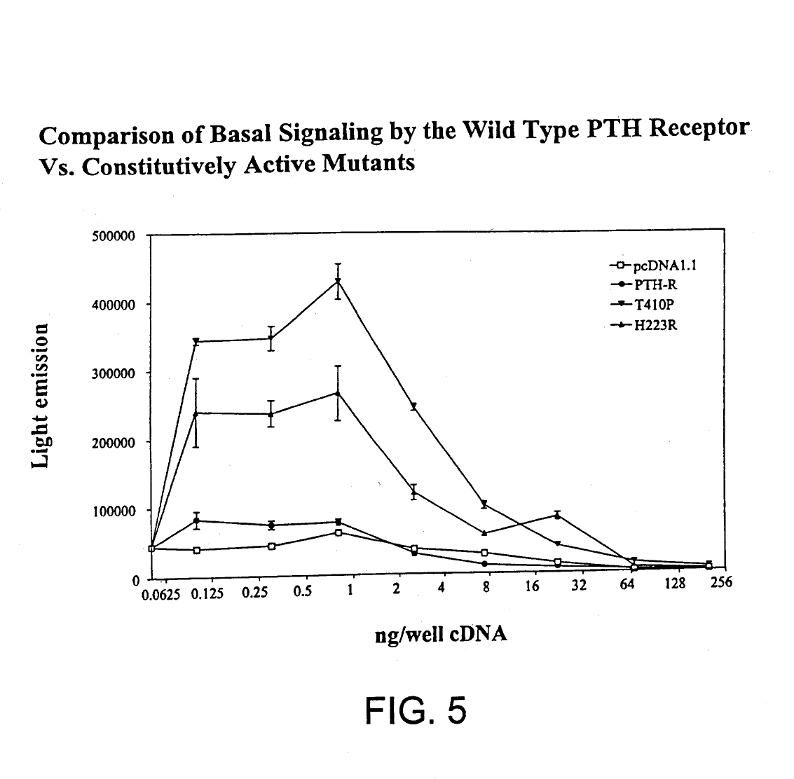

A marked increase in basal signaling was observed with expression of the cDNA encoding the PTH receptor with either activating point mutation (FIG. 5, H223R or T410P).

### EXAMPLE 4

Constitutively Active Mu Opioid Receptor

Wild type mu opioid receptor (Gi coupled) and a receptor mutant which is constitutively active (mu OR-MO1) were assessed over a wide range of DNA co-transfection amounts. DNA “dose response” curves were used to demonstrate constitutive activity independent of ligand stimulation. Each well was co-transfected with 35 ng reporter+7 ng Gq5i overnight. Cells were assayed the following day using the LucLite Luciferase Assay Kit (Packard).

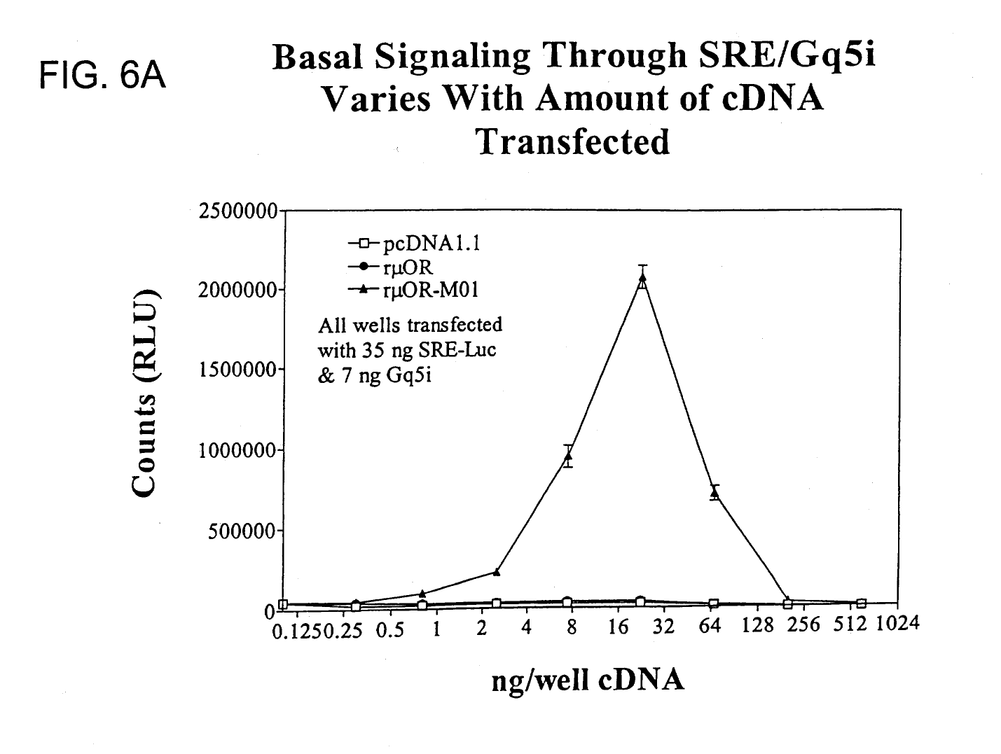

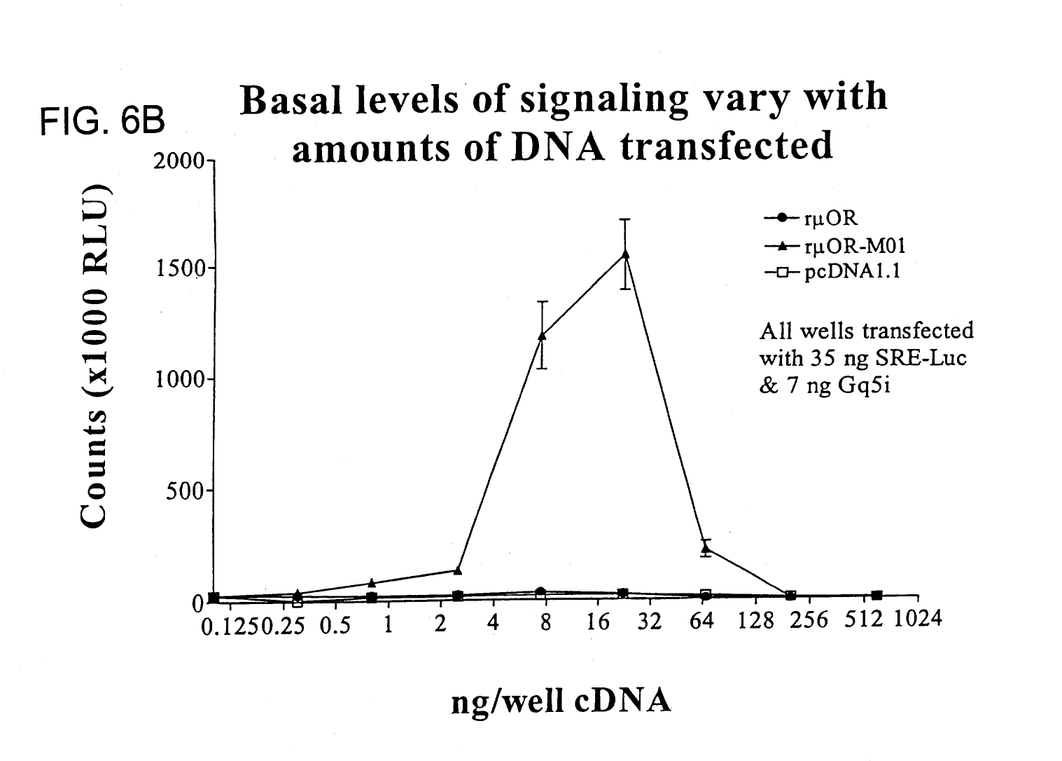

Over a wide range of transfected cDNA (FIGS. 6A-B), the wild type basal (unstimulated) signaling approximated signaling in cells transfected with the empty expression vector pcDNA 1.1. In contrast, the constitutively active mutant induced signaling that was significantly elevated above wild type values.

### EXAMPLE 5

Co-Expression of a Constitutively Active Receptor With Another Receptor Non-Specifically Reduces Signaling of the Constitutively Active Receptor

This example illustrates that co-expression of a constitutively active first receptor with a different second receptor may non-specifically reduce signaling induced by the first receptor, regardless of the basal activity or the signaling mechanism of the second receptor. For each experiment, each well was transfected with 35 ng Sms-Luc and 2.5 ng MC4-M03 (a constitutively active variant of MC4-R), as well as second receptor cDNA or control DNA. Transfection was overnight. Cells were then stimulated (+or−ligand) overnight in the presence of protease inhibitor. Cells were assayed using the LucLite Luciferase Assay Kit from Packard.

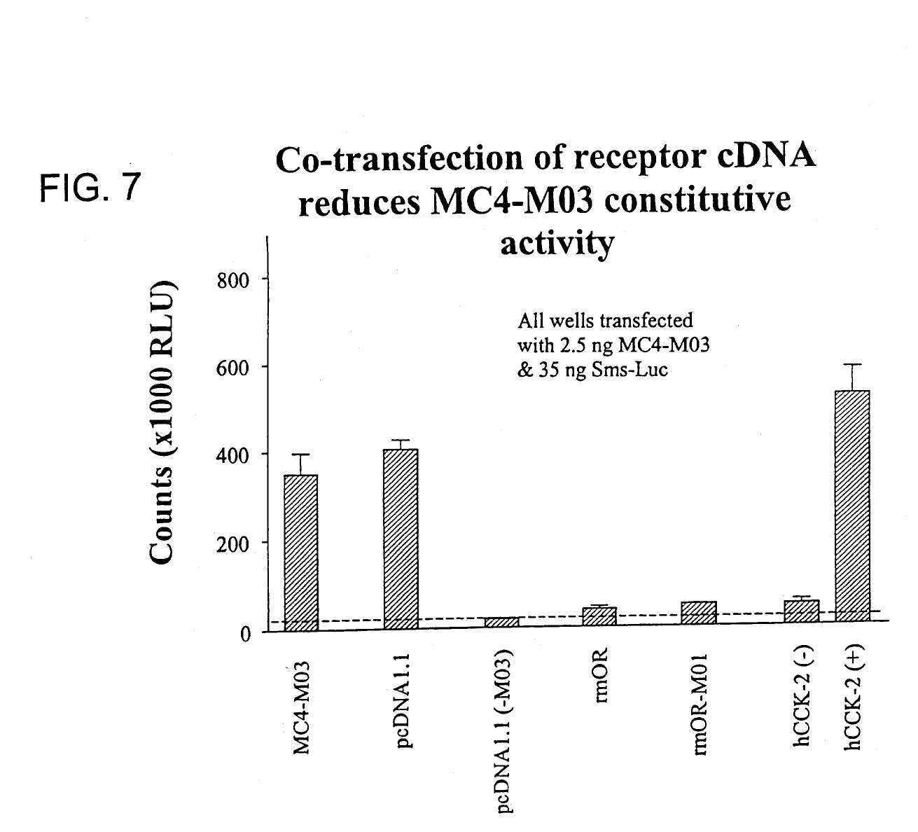

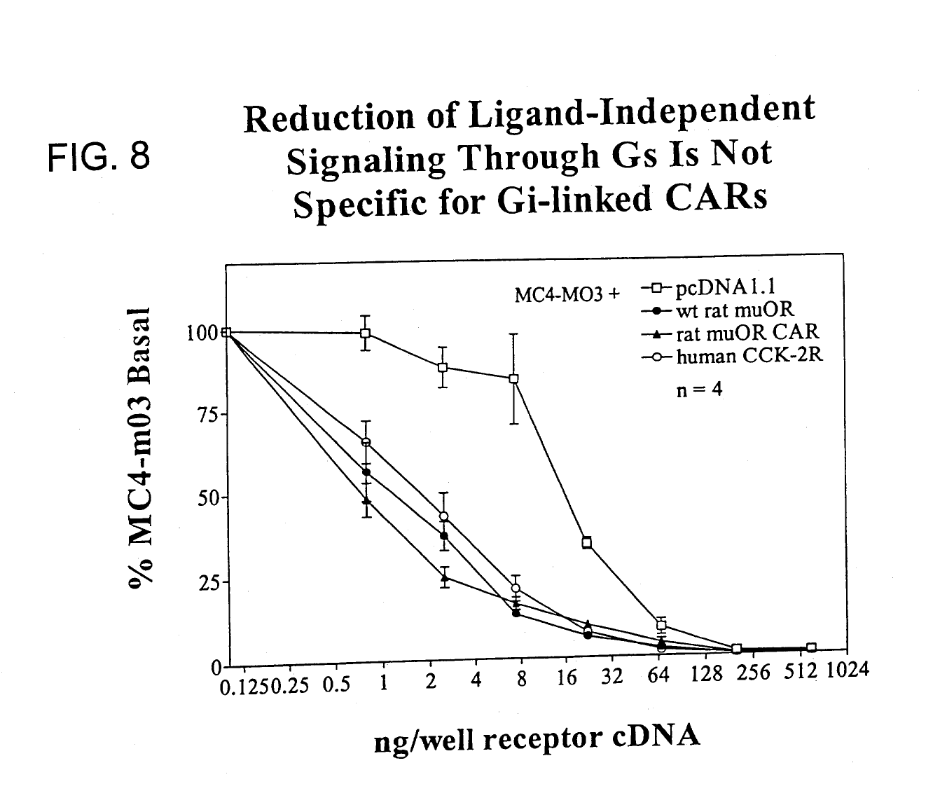

Expression of a constitutively active MC4 receptor mutant (MC4-M03) lead to a high level of Gs-mediated basal signaling, compared to the empty expression vector, pcDNA1.1 (as also demonstrated in Example 2) (see FIG. 7). Co-expression of either the wild type Mu opioid receptor (rmOR; Gi coupled however with no basal activity, see Example 4), a constitutively active Mu opioid receptor mutant (rmOR-M01; predicted to be a strong inhibitor of Gs mediated signaling due to basal Gi function, see Example 4), or the CCK-2 receptor (hCCK-2; predicted to have no basal activity and also work through a different, Gq-mediated, mechanism than MC4-M03, see example 1) all virtually abolish MC4-M03 induced basal signaling. Thus, reduction of MC4-M03 function in the presence of other receptors in this assay occurs through mechanisms that are not indicative of the signaling properties of the other receptors.

### EXAMPLE 6

Inhibition of a Constitutively Active Receptor by Co-Expression of a Second Receptor Cannot be Attributed to Specific Functional Properties of the Second Receptor

This example illustrates that inhibition of a constitutively active first receptor by co-expression of a different second receptor cannot be attributed to specific functional properties of the second receptor, even if the latter is assessed over a wide concentration range. For each experiment, wells were co-transfected with 35 ng Sms-Luc and 2.5 ng MC4-M03 (a constitutively active variant of MC4-R), as well as specified second receptor cDNA or control DNA. Transfection was overnight. Cells were then incubated overnight to assess the level of ligand independent signaling. Cells were assayed using the LucLite Luciferase Assay Kit from Packard.

Enhanced basal signaling of a constitutively active MC4 receptor mutant (MC4-M03) is gradually reduced by increasing co-expression of either a wild type Mu opioid receptor (Gi coupled, no basal activity), a constitutively active Mu opioid receptor mutant (MuOR CAR, ligand-independent Gi coupling), or a CCK-2 receptor (no basal activity, Gq coupled). Concentration-dependent inhibition of signaling by either of these second receptors is similar, indicating that the degree of observed inhibition does not correlate with either the signaling pathway coupled to the second receptor or its constitutive activity. In fact, even co-expression of the empty expression vector, pcDNA1.1, concentration dependently inhibits MC4-M03 induced signaling (although at higher DNA concentrations), suggesting that inhibition at least in part reflects a receptor-independent, non-specific process.

Other Embodiments

All publications and patent applications mentioned in this specification are herein incorporated by reference to the same extent as if each independent publication or patent application was specifically and individually indicated to be incorporated by reference. While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follow in the scope of the appended claims.

1. A method of identifying a receptor with altered signaling, said method comprising:
(a) co-transfecting a first host cell with:
(i) an expression vector, said expression vector comprising a promoter operably linked to a candidate receptor, and
(ii) a reporter construct, said reporter construct comprising a response element and a promoter operably linked to a reporter gene, said response element being sensitive to a signal induced by said receptor;
(b) co-transfecting a second host cell with said reporter construct and a negative control vector;
(c) measuring the level of expression of said reporter construct in said first host cell and in said second host cell, at varying concentrations of said reporter construct or at varying concentrations of said expression vector or said negative control vector, whereby dose-response curves are generated for said expression of said reporter construct in said first and said second host cells; and
(d) identifying said candidate receptor as a receptor with altered signaling by its ability to increase or decrease said level of expression in the first host cell compared to said level of expression in the second host cell over a range of at least two different concentrations of said reporter construct, said negative control vector, or said expression vector.

2. The method of claim 1, wherein said reporter construct is selected from the group consisting of a luciferase construct, a beta-galactosidase construct, and a chloramphenicol acetyl transferase construct.

3. The method of claim 2, wherein reporter construct is a luciferase construct.

4. The method of claim 1, wherein said response element is selected from the group consisting of the somatostatin promoter, the serum response element, and the cAMP response element.

5. The method of claim 1, wherein said receptor with altered signaling is selected from the group consisting of a constitutively active receptor, a hypersensitive receptor, a hyposensitive receptor, a non-functional receptor, a silent receptor, and a partially silent receptor.

6. The method of claim 1, wherein said receptor with altered signaling is a G protein-coupled receptor.

7. The method of claim 6, wherein said G protein-coupled receptor is coupled to a G protein selected from the group consisting of Gαq, Gαs, Gαi, and Go.

8. The method of claim 6, said method further comprising:
in step (a), co-transfecting said first host cell with a second expression vector, said second expression vector comprising a promoter operably linked to a chimeric G protein, wherein said chimeric G protein is capable of receiving a signal from said G protein-coupled receptor and increasing the expression of said reporter construct; and
in step (b), co-transfecting said second host cell with said second expression vector.

9. The method of claim 8, wherein said chimeric G protein is selected from the group consisting of Gq5i, Gq5o, Gq5z, Gq5s, Gs5q, and G13Z.

10. The method of claim 1, wherein said receptor with altered signaling is selected from the group consisting of a transmembrane receptor, a nuclear receptor, and a steroid hormone receptor.

11. The method of claim 1, wherein said receptor with altered signaling is selected from the group consisting of a mutant receptor and a polymorphic receptor.

12. The method of claim 1, wherein said range is over at least three different concentrations of said reporter construct or said expression vector.

13. The method of claim 1, wherein said range is over at least five different concentrations of said reporter construct or said expression vector.

14. The method of claim 1, wherein said signaling is ligand dependent signaling.

15. The method of claim 1, wherein said signaling is ligand independent signaling.

16. A method of identifying a G protein-coupled receptor with altered signaling, said method comprising:
(a) co-transfecting a first host cell with:
(i) a reporter construct, said reporter construct comprising a G protein response element and a promoter operably linked to a reporter gene,
(ii) a first expression vector, said first expression vector comprising a promoter operably linked to a candidate G protein-coupled receptor, and
(iii) a second expression vector, said second expression vector comprising a promoter operably linked to a chimeric G protein, wherein said chimeric G protein is capable of receiving a signal from said candidate G protein-coupled receptor and increasing the expression of said reporter construct;
(b) co-transfecting a second host cell with said reporter construct, said second expression vector, and a negative control vector; and
(c) measuring the level of expression of said reporter construct in said first host cell and said second host cell, wherein an increased or decreased level of expression in the first host cell compared to the second host cell identifies said candidate receptor as a G protein-coupled receptor with altered signaling.

17. The method of claim 16, wherein said chimeric G protein comprises a G protein with the C-terminal 3 amino acids changed to those of another G protein.

18. The method of claim 16, wherein chimeric G protein is selected from the group consisting of Gq5i, Gq5o, Gq5z, Gq5s, Gs5q, and G13Z.

19. The method of claim 16, wherein said reporter construct is selected from the group consisting of a luciferase construct, a beta-galactosidase construct, and a chloramphenicol acetyl transferase construct.

20. The method of claim 19, wherein reporter construct is a luciferase construct.

21. The method of claim 16, wherein said response element is selected from the group consisting of the somatostatin promoter, the serum response element, and the cAMP response element.

22. The method of claim 16, wherein said G protein coupled receptor is selected from the group consisting of a constitutively active receptor, a hypersensitive receptor, a hyposensitive receptor, a non-functional receptor, a silent receptor, and a partially silent receptor.

23. The method of claim 16, wherein said G protein-coupled receptor is coupled to a G protein selected from the group consisting of Gαq, Gαs, GαI, and Go.

24. The method of claim 16, wherein said signaling is ligand dependent signaling.

25. The method of claim 16, wherein said signaling is ligand independent signaling.

26. The method of claim 16, wherein said receptor with altered signaling is selected from the group consisting of a mutant receptor and a polymorphic receptor.
